(19) United States
(12) Patent Application Publication
(10) Pub. No.: US 2013/0190223 A1
(43) Pub. Date: Jul. 25, 2013

(54) NONIMMUNOSUPPRESSIVE CYCLOSPORINE ANALOGUE MOLECULES

(71) Applicants: Alexander Hegmans, Edmonton (CA); Bruce W. Fenske, Edmonton (CA); David P. Czajkowski, Edmonton (CA); Darren R. Ure, Edmonton (CA); Shin Sugiyama, Edmonton (CA); Dan J. Trepanier, Edmonton (CA); David H. McGlade, Edmonton (CA); Mark D. Abel, Edmonton (CA)

(72) Inventors: Alexander Hegmans, Edmonton (CA); Bruce W. Fenske, Edmonton (CA); David P. Czajkowski, Edmonton (CA); Darren R. Ure, Edmonton (CA); Shin Sugiyama, Edmonton (CA); Dan J. Trepanier, Edmonton (CA); David H. McGlade, Edmonton (CA); Mark D. Abel, Edmonton (CA)

(73) Assignee: ISOTECHNIKA PHARMA INC., Edmonton, AB (CA)

(21) Appl. No.: 13/056,616

(22) PCT Filed: Jul. 3, 2009
(86) PCT No.: PCT/CA2009/000917

§ 371 Date: Mar. 22, 2011

Related U.S. Application Data
(60) Provisional application No. 61/137,522, filed on Jul. 30, 2008.
(60) Provisional application No. 61/084,999, filed on Jul. 31, 2008.

(51) Int. Cl. C07K 7/64

(52) U.S. Cl. CPC C07K 7/645
USPC 514/1.7; 530/317; 514/20.5; 514/3.8; 514/4.3; 514/16.6; 514/19.3; 514/19.4; 514/17.8; 514/17.7; 514/18.2

(57) ABSTRACT

The compounds of the present invention are non-immunosupressive cyclosporine analogue molecules that are able to bind cyclophilin. Said compounds include a modified side chain of amino acid I of cyclosporin A, consisting of an oxyalkyl having substituents R′, R1 and R2, where R′ is H or Acetyl; R1 is a saturated or unsaturated straight chain or branched aliphatic carbon chain; and R2 may be a hydrogen; a unsubstituted, N substituted or NN disubstituted amide; a N substituted or unsubstituted acyl protected amine; a carboxylic acid; a N substituted or unsubstituted amine; a nitrile; a ester; a ketone; a hydroxy, dihydroxy, trihydroxy or polyhydroxy alkyl; or a substituted or unsubstituted aryl.

This application claims Convention priority from U.S. Patent Application No. 61/137,522, filed Jul. 30, 2008, and U.S. Patent Application No. 61/084,999, filed Jul. 31, 2008, said applications being wholly incorporated herein by reference.

### FIELD OF THE INVENTION

The present invention relates to novel analogs of molecules belonging to the cyclosporine family and in particular of Cyclosporine A (CsA), that have reduced or no immunosuppressive activity and bind cyclophilin (CyP).

### BACKGROUND OF THE INVENTION

Cyclosporines are members of a class of cyclic polypeptides having potent immunosuppressant activity. At least some of these compounds, such as Cyclosporine A (CsA), are produced by the species Tolypocladium inflatum as secondary metabolites. CsA is a potent immunosuppressive agent that has been demonstrated to suppress humoral immunity and cell-mediated immune reactions, such as allograft rejection, delayed hypersensitivity, experimental allergic encephalomyelitis, Freund's adjuvant arthritis and graft versus host disease. It is used for the prophylaxis of organ rejection in organ transplants; for the treatment of rheumatoid arthritis; and for the treatment of psoriasis.

Although a number of compounds in the cyclosporine family are known, CsA is perhaps the most widely used medically. The immunosuppressive effects of CsA are related to the inhibition of T-cell mediated activation events. Immunosuppression is accomplished by the binding of cyclosporine to a ubiquitous intracellular protein called cyclophilin (CyP). This complex, in turn, inhibits the calcium and calmodulin-dependent serine-threonine phosphatase activity of the enzyme calcineurin. Inhibition of calcineurin prevents the activation of transcription factors, such as NFAT_{p/c} and NF-κB, which are necessary for the induction of cytokine genes (IL-2, IFN-γ, IL-4, and GM-CSF) during T-cell activation.

Since the original discovery of cyclosporine, a wide variety of naturally occurring cyclosporines have been isolated and identified. Additionally, many cyclosporines that do not occur naturally have been prepared by partial or total synthetic means, and by the application of modified cell culture techniques. Thus, the class comprising cyclosporines is substantial and includes, for example, the naturally occurring cyclosporines A through Z; various non-naturally occurring cyclosporine derivatives; artificial or synthetic cyclosporines including the dihydro- and iso-cyclosporines; derivatized cyclosporines (for example, either the 3′-O-atom of the MeBmt residue may be acylated, or a further substituent may be introduced at the sarcosyl residue at the 3-position); cyclosporines in which the MeBmt residue is present in isomeric form (e.g., in which the configuration across positions 6′ and 7′ of the MeBmt residue is cis rather than trans); and cyclosporines wherein variant amino acids are incorporated at specific positions within the peptide sequence.

Cyclosporine analogues containing modified amino acids in the 1-position are disclosed in WO 99/18120 and WO 03/033527, which are incorporated herein by reference in their entirety. These applications describe a cyclosporine derivative known as “ISA_{TX}247” or “ISA247” or “ISA.” This analog is structurally identical to CsA, except for modification at the amino acid-1 residue. Applicants have previously discovered that certain mixtures of cis and trans isomers of ISA247, including mixtures that are predominantly comprised of trans ISA247, exhibited a combination of enhanced immunosuppressive potency and reduced toxicity over the naturally occurring and presently known cyclosporines.

Cyclosporine has three well established cellular targets; calcineurin, the CyP isoforms (which includes but is not limited to CyP-A, CyP-B and CyP-D), and P-glycoprotein (PgP). The binding of cyclosporine to calcineurin results in significant immunosuppression and is responsible for its traditional association with transplantation and autoimmune indications.

### The Cyclophilin Family

CyPs (Enzyme Commission (EC) number 5.1.2.8) belong to a group of proteins that have peptidyl-prolyl cis-trans isomerase activity; such proteins are collectively known as immunophilins and also include the FK-506-binding proteins and the parvulins. CyPs are found in all cells of all organisms studied, in both prokaryotes and eukaryotes and are structurally conserved throughout evolution. There are 7 major CyPs in humans, namely CyP-A, CyP-B, CyP-C, CyP-D, CyP-E, CyP-40, and CyP-NK (first identified from human natural killer cells), and a total of 16 unique proteins (Galat A. Peptidylprolyl cis/trans isomerases (immunophilins): biological diversity-targets-functions. Curr Top Med Chem 2003, 3:1315-1347; Waldmeier P C et al. Cyclophilin D as a drug target. Curr Med Chem 2003, 10:1485-1506).

The first member of the CyPs to be identified in mammals was CyP-A. CyP-A is an 18-kDa cytosolic protein and is the most abundant protein for CsA binding. It is estimated that CyP-A makes up 0.6% of the total cytosolic protein (Mikol V et al. X-ray structure of monmeric cyclophilin A-cycloporin A crystal complex at 2.1 A resolution. J. Mol. Biol. 1993, 234:1119-1130; Galat A et al. Metcalfe S M. Peptidylproline cis/trans isomerases. Prog. Biophys. Mol. Biol. 1995, 63:67-118).

### Cellular Location of Cyclophilins

CyPs can be found in most cellular compartments of most tissues and encode unique functions. In mammals, CyP-A and CyP-40 are cytosolic whereas CyP-B and CyP-C have amino-terminal signal sequences that target them to the endoplasmic reticulum protein secretory pathway (reviewed in Galat, 2003; Dornan J et al. Structures of immunophilins and their ligand complexes. Curr Top Med Chem 2003, 3:1392-1409). CyP-D has a signal sequence that directs it to the mitochondria (Andreeva, 1999; Hamilton G S et al. Immunophilins: beyond immunosuppression. J Med Chem 1998, 41:5119-5143); CyP-E has an amino-terminal RNA-binding domain and is localized in the nucleus (Mi H et al. A nuclear RNA-binding cyclophilin in human T cells. FEBS Lett 1996, 398:201-205) and CyP-40 has TPRs and is located in the cytosol (Kieffer L J et al. Cyclophilin-40, a protein with homology to the P59 component of the steroid receptor complex. Cloning of the cDNA and further characterization. J Biol Chem 1993, 268:12303-12310). Human CyP-NK is the largest CyP, with a large, hydrophilic and positively charged carboxyl terminus, and is located in the cytosol (Anderson S K et al. A cyclophilin-related protein involved in the function of natural killer cells. Proc Natl Acad Sci USA 1993, 90:542-546; Rinfret A et al. The N-terminal cyclophilin-homologous domain of a 150-kilodalton tumor recognition molecule exhibits both peptidylprolyl cis-transisomerase and chaperone activities. Biochemistry 1994, 33:1668-1673)

### Function and Activity of the Cyclophilins

CyPs are multifunctional proteins that are involved in many cellular processes.

Because CyPs were highly conserved throughout evolution, this suggests an essential role for CyPs. Initially, it was found that CyPs have the specific enzymatic property of catalyzing cis-trans isomerization of peptidyl-prolyl bonds (Galat, 1995; Fisher G A, Halsey J, Hausforff J, et al. A phase I study of paclitaxel (taxol) (T) in combination with SDZ valspodar, a potent modulator of multidrug resistance (MDR). Anticancer Drugs. 1994; 5(Suppl 1): 43). Thus, CyPs are called peptidyl-prolyl-cis-trans isomerase (PPlase), which can act as an acceleration factor in the proper folding of newly synthesized proteins, PPlases are also involved in repairing damaged proteins due to environmental stresses including thermal stress, ultraviolet irradiation, changes in the pH of the cell environment, and treatment with oxidants. This function is known as molecular chaperoning activity. LYao Q et al. Roles of Cyclophilins in Cancers and Other Organs Systems. World J. Surg. 2005, 29: 276-280)

In addition, the PPlase activity of CyPs has recently been shown to be involved in diverse cellular processes, including intracellular protein trafficking (Andreeva L et al. Cyclophilins and their possible role in the stress response. Int J Exp Pathol 1999, 80:305-315, Caroni P et al. New member of the cyclophilin family associated with the secretory pathway. J Biol Chem 1991, 266:10739-42), mitochondrial function (Halestrap A P et al. CsA binding to mitochondrial cyclophilin inhibits the permeability transition pore and protects hearts from ischaemia/reperfusion injury. Mol Cell Biochem 1997, 174:167-72; Connern C P, Halestrap A P. Recruitment of mitochondrial cyclophilin to the mitochondrial inner membrane under conditions of oxidative stress that enhance the opening of a calcium-sensitive non-specific channel. Biochem J 1994, 302:321-4), pre-mRNA processing (Bourquin J P et al. A serine/argininerich nuclear matrix cyclophilin interacts with the Cterminal domain of RNA polymerase II. Nucleic Acids Res 1997, 25:2055-61), and maintenance of multiprotein complex stability (Andreeva, 1999).

Cyclosporine binds with nanomolar affinity to CyP-A via contacts within the hydrophobic pocket (Colgan J et al. Cyclophilin A-Deficient Mice Are Resistant to Immunosuppression by Cyclosporine. The Journal of Immunology 2005, 174: 6030-6038, Mikol, 1993) and inhibits PPlase activity. However, this effect is thought to be irrelevant for the immunosuppression. Rather, the complex between CsA and CyP-A creates a composite surface that binds to and prevents calcineurin from regulating cytokine gene transcription (Friedman J et al. Two cytoplasmic candidates for immunophilin action are revealed by affinity for a new cyclophilin: one in the presence and one in the absence of CsA. Cell 1991, 66: 799-806; Liu J et al. Calcineurin is a common target of cyclophilin-CsA and FKBP-FK506 complexes. Cell 1991, 66: 807-815).

### Homology of the Cyclophilins

CyP-A, the prototypical member of the family, is a highly conserved protein in mammalian cells (Handschumacher R E et al. Cyclophilin: a specific cytosolic binding protein for CsA. Science 1984, 226: 544-7). Sequence homology analysis of human CyP-A shows that it is highly homologous to human CyP-B, CyP-C, and CyP-D (Harding M W, Handschumacher R E, Speicher D W. Isolation and amino acid sequence of cyclophilin. J Biol Chem 1986, 261:8547-55). The cyclosporine binding pocket of all CyPs is formed by a highly conserved region of approximately 109 amino acids. Of the known CyPs, CyP-D has the highest homology to CyP-A. In fact, in this region the sequence identity is 100% between CyP-A and CyP-D (Waldmeier 2003; Kristal B S et al. The Mitochondrial Permeability Transition as a Target for Neuroprotection. Journal of Bioenergetics and Biomembranes 2004, 36(4); 309-312). Therefore, CyP-A affinity is a very good predictor of CyP-D affinity, and visa versa (Hansson M J et al. The Nonimmunosuppressive Cyclosporine analogues NIM811 and UNIL025 Display Nanomolar Potencies on Permeability Transition in Brain-Derived Mitochondria. Journal of Bioenergetics and Biomembranes, 2004, 36(4): 407-413). This relationship has been repeatedly demonstrated empirically with Cyclosporine analogues (Hansson, 2004; Ptak Rg et al. Inhibition of Human Immunodeficiency Virus Type 1 Replication in Human Cells by Debio-025, a Novel Cyclophilin Binding Agent Antimicrobial Agents and Chemotherapy 2008: 1302-1317; Millay D P et al. Genetic and pharmacologic inhibition of mitochondrial dependent necrosis attenuates muscular dystrophy. Nature Medicine 2008, 14(4): 442-447; Harris R et al. The Discovery of Novel Non-Immunosuppressive Cyclosporine Ethers and Thioethers With Potent HCV Activity. Poster #1915, 59th Annual Meeting of the American Association for the Study of Liver Diseases (AASLD), 2008). The sequence homology across the CyPs suggests that all CyPs are potential targets for Cyclosporine analogues. Because of the multitude of cellular processes in which the CyPs are involved, this further suggests that CsA analogues which retain significant binding to CyP can be useful in the treatment of many disease indications.

### Cyclophilin Mediated Diseases

### Human Immunodeficiency Virus (HIV):

HIV is lentivirus of the retrovirus family and serves as an example fo the involvement of CyP in the process of infection and replication of certain viruses. CyP-A was established more than a decade ago to be a valid target in anti-HIV chemotherapy (Rosenwirth B A et al. Cyclophilin A as a novel target in anti-HIV-1 chemotherapy. Int Antivir. News 1995, 3:62-63). CyP-A fulfills an essential function early in the HIV-1 replication cycle. It was found to bind specifically to the HIV-1 Gag polyprotein (Luban J K L et al. Human immunodeficiency virus type 1 Gag protein binds to cyclophilins A and B. Cell 1993, 73: 1067-1078). A defined amino acid sequence around G89 and P90 of capsid protein p24 (CA) was identified as the binding site for CyP-A (Bukovsky A A A et al. Transfer of the HIV-1 cyclophilin-binding site to simian immunodeficiency virus from Macaca mulatta can confer both cyclosporine sensitivity and cyclosporine dependence. Proc. Natl. Acad. Sci. USA 1997, 94: 10943-10948; Gamble T R F et al. Crystal structure of human cyclophilin A bound to the amino-terminal domain of HIV-1 capsid. Cell 1996, 87: 1285-1294). The affinity of CyP-A for CA promotes the incorporation of CyP-A into the virion particles during assembly (Thali M A et al. Functional association of cyclophilin A with HIV-1 virions. Nature 1994, 372: 363-365). Experimental evidence indicates that the CyP-A-CA interaction is essential for HIV-1 replication; inhibition of this interaction impairs HIV-1 replication in human cells (Hatziioannou T D et al. Cyclophilin interactions with incoming human immunodeficiency virus type 1 capsids with opposing effects on infectivity in human cells. J. Virol. 2005, 79: 176-183; Steinkasserer A R et al. Mode of action of SDZ NIM 811, a nonimmunosuppressive CsA analog with activity against human immunodeficiency virus type 1 (HIV-1): interference with early and late events in HIV-1 replication. J. Virol 1995, 69: 814-824). The step in the viral replication cycle where CyP-A is involved was demonstrated to be an event after penetration of the virus particle and before integration of the double-stranded viral DNA into the cellular genome (Braaten D E K et al. Cyclophilin A is required for an early step in the life cycle of human immunodeficiency virus type 1 before the initiation of reverse transcription. J. Virol 1996 70: 3551-3560, Mlynar E D et al. The non-immunosuppressive CsA analogue SDZ NIM 811 inhibits cyclophilin A incorporation into virions and virus replication in human immunodeficiency virus type 1-infected primary and growth-arrested T cells. J. Gen. Virol 1996, 78: 825-835; Steinkasserer, 1995). The anti-HIV-1 activity of CsA was first reported in 1988 (Weinberg M A et al. The effect of CsA on infection of susceptible cells by human immunodeficiency virus type 1. Blood 1998, 72: 1904-1910). Evaluation of CsA and many derivatives for inhibition of HIV-1 replication revealed that nonimmunosuppressive CsA analogs had anti-HIV-1 activities equal to or even superior to those of immunosuppressive analogs (Bartz S R E et al. Inhibition of human immunodeficiency virus replication by nonimmunosuppressive analogs of CsA. Proc. Natl. Acad. Sci. USA 1995, 92: 5381-5385, Billich A F et al. Mode of action of SDZ NIM 811, a nonimmunosuppressive CsA analog with activity against human immunodeficiency virus (HIV) type 1: interference with HIV protein-cyclophilin A interactions. J. Virol 1995, 69: 2451-2461; Ptak, 2008).

### Inflammation

Inflammation in disease involves the influx of Leukocytes (white blood cells) to the area of affection. The leukocytes are drawn to the area by chemokines, a family of chemoattracting agents. In vitro studies have shown that extracellular CyP-A is a potent chemoattractant for human leukocytes and T cells (Kamalpreet A et al. Extracellular Cyclophilins Contribute to the Regulation of Inflammatory Responses Journal of Immunology 2005; 175: 517-522; Yurchenko V G et al. Active-site residues of cyclophilin A are crucial for its signaling activity via CD147. J. Biol. Chem. 2002; 277: 22959-22965; Xu Q M C et al. Leukocyte chemotactic activity of cyclophilin. J. Biol. Chem. 1992; 267: 11968-11971; Allain F C et al. Interaction with glycosaminoglycans is required for cyclophilin B to trigger integrin-mediated adhesion of peripheral blood T lymphocytes to extracellular matrix. Proc. Natl. Acad. Sci. USA 2002; 99: 2714-2719). Furthermore, CyP-A can induce a rapid inflammatory response, characterized by leukocyte influx, when injected in vivo (Sherry B N et al. Identification of cyclophilin as a proinflammatory secretory product of lipopolysaccharide-activated macrophages. Proc. Natl. Acad. Sci. USA 1992; 89: 3511-3515). CyP-A is ubiquitously distributed intracellularily, however, during the course of inflammatory responses, CyP-A is released into extracellular tissue spaces by both live and dying cells (Sherry, 1992). Indeed, elevated levels of CyP-A have been reported in several different inflammatory diseases, including sepsis, rheumatoid arthritis, and vascular smooth muscle cell disease (Jin Z G et al. Cyclophilin A is a secreted growth factor induced by oxidative stress. Circ. Res. 2000; 87: 789-796; Teger, 1997; Billich, 1997). In the case of rheumatoid arthritis, a direct correlation between levels of CyP-A and the number of neutrophils in the synovial fluid of rheumatoid arthritis patients was reported (Billich, 1997).

### Cancer

CyP-A has recently been shown to be over-expressed in many cancer tissues and cell lines, including but not limited to small and non-small cell lung, bladder, hepatocellular, pancreatic and breast cancer (Li, 2006; Yang H et al. Cyclophilin A is upregulated in small cell lung cancer and activates ERK1/2 signal. Biochemical and Biophysical Research Communications 2007; 361: 763-767; Campa, 2003). In cases where exogenous CyP-A was supplied this was shown to stimulate the cancer cell growth (Li, 2006; Yang, 2007) while CsA arrested the growth (Campa, 2003). Most recently it has been demonstrated the CyP (A and B) is intricately involved in the biochemical pathway allowing growth of human breast cancer cells and that CyP knockdown experiments decreased the cancer cell growth, proliferation and motility (Fang F et al. The expression of Cyclophilin B is Associated with Malignant Progression and Regulation of Genes Implicated in the Pathogenesis of Breast Cancer. The American Journal of Pathology 2009; 174(1): 297-308; Zheng J et al. Prolyl Isomerase Cyclophilin A Regulation of Janus-Activated Kinase 2 and the Progression of Human Breast Cancer. Cancer Research 2008; 68 (19): 7769-7778). Most interestingly, CsA treatment of mice xenografted with breast cancer cells induced tumor necrosis and completely inhibited metastasis (Zheng, 2008). The researchers conclude that “Cyclophilin B action may significantly contribute to the pathogenesis of human breast cancer” and that “cyclophilin inhibition may be a novel therapeutic strategy in the treatment of human breast cancer” (Fang, 2009; Zheng, 2008).

### Hepatitis C

Hepatitis C Virus (HCV) is the most prevalent liver disease in the world and is considered by the World Health Organization as an epidemic. Because HCV can infect a patient for decades before being discovered, it is often called the “silent” epidemic. Studies suggest that over 200 million people worldwide are infected with HCV, an overall incidence of around 3.3% of the world's population. In the US alone, nearly 4 million people are or have been infected with HCV and of these; 2.7 million have an ongoing chronic infection. All HCV infected individuals are at risk of developing serious life-threatening liver diseases. Current standard therapy for chronic hepatitis C consists of the combination of pegylated interferon in combination with ribavirin, both generalized anti-viral agents (Craxi A et al. Clinical trial results of peginterferons in combination with ribavirin. Semin Liver Dis 2003; 23(Suppl 1): 35-46). Failure rate for the treatment is approximately 50% (Molino B F. Strategic Research Institute: 3^{rd} annual viral hepatitis in drug discovery and development world summit 2007. AMRI Technical Reports; 12(1)).

It has recently been demonstrated that CyP-B is critical for the efficient replication of the hepatitis C virus (HCV) genome (Watashi K et al. Cyclophilin B Is a Functional Regulator of Hepatitis C Virus RNA Polymerase. Molecular Cell 2005, 19: 111-122). Viruses depend on host-derived factors such as CyP-B for their efficient genome replication. CyP-B interacts with the HCV RNA polymerase NS5B to directly stimulate its RNA binding activity. Both the RNA interference (RNAi)-mediated reduction of endogenous CyP-B expression and the induces loss of NS5B binding to CyP-B decreases the levels of HCV replication. Thus, CyP-B functions as a stimulatory regulator of NS5B in HCV replication machinery. This regulation mechanism for viral replication identifies CyP-B as a target for antiviral therapeutic strategies. Unlike other HCV treatments, cyclophilin inhibition does not directly target the HCV virus. It is therefore thought that resistance to CyP binding drugs will occur more slowly than current HCV treatment drugs (Manns M P, et al. The way forward in HCV treatment-finding the right path. Nature Reviews Drug Discovery 2007; 6: 991-1000). In addition, by interfering at the level of host-viral interaction, CyP inhibition may open the way for a novel approach to anti-HCV treatment that could be complementary, not only to interferon-based treatment, but also to future treatments that directly target HCV replication enzymes such as protease and polymerase inhibitors (Flisiak R, Dumont J M, Crabbé R. Cyclophilin inhibitors in hepatitis C viral infection. Expert Opinion on Investigational Drugs 2007, 16(9): 1345-1354). Development of new anti-HCV drugs effecting HCV viral replication has been significantly impeded by the lack of a suitable laboratory HCV model. This obstacle has only recently been overcome by the development of several suitable cell culture models (Subgenomic HCV Replicon Systems) and a mouse model containing human liver cells (Goto K, et al. Evaluation of the anti-hepatitis C virus effects of cyclophilin inhibitors, CsA, and NIM811. Biochem Biophys Res Comm 2006; 343: 879-884; Mercer D F, et al. Hepatitis C virus replication in mice with chimeric human livers. Nat Med 2001; 7: 927-933). Cyclosporine has recently demonstrated anti-HCV activity in screening models and in small clinical trials (Watashi K, et al. CsA suppresses replication of hepatitis C virus genome in cultured hepatocytes. Hepatology 2003; 38:1282-1288; Inoue K, Yoshiba M. Interferon combined with cyclosporine treatment as an effective countermeasure against hepatitis C virus recurrence in liver transplant patients with end-stage hepatitis C virus related disease. Transplant Proc 2005; 37:1233-1234).

### Muscular Degenerative Disorders

CyP-D is an integral part of the mitochondrial permeability transition pore (MTP) in all cells. The function of the MTP pore is to provide calcium homeostasis within the cell. Under normal conditions the opening and closing of the MTP pore is reversible. Under pathological conditions which involve an excessive calcium influx into the cell, this overloads the mitochondria and induces an irreversible opening of the MPT pore, leading to cell death or apoptosis. CsA has been reported to correct mitochondrial dysfunction and muscle apoptosis in patients with Ullrich congenital muscular dystrophy and Bethlam myopathy [(Merlini L et al. CsA corrects mitochondrial dysfunction and muscle apoptosis in patients with collagen VI myopathies. PNAS 2008; 105(13): 5225-5229]. CsA has been demonstrated in vitro to dose dependently inhibit mPTP opening in isolated cardiac mitochondria, thereby preventing apoptosis and allowing the cell precious time for repair (Gomez L et al. Inhibition of mitochondrial permeability transition improves functional recovery and reduces mortality following acute myocardial infarction in mice Am J Physiol Heart Circ Physiol 2007, 293: H1654-H1661). A clinical study in 58 patients who presented with acute myocardial infarction demonstrated that administration of CsA at the time of reperfusion was associated with a smaller infarct than that seen with placebo (Piot C et al. Effect of Cyclosporine on Reperfusion Injury in Acute Myocardial Infarction. New England Journal of Medicine 2008; 395(5): 474-481)).

### Chronic Neurodegenerative Diseases

CsA can act as a neuroprotective agent in cases of acute cerebral ischemia and damage, as a result of head trauma (Keep M, et al. Intrathecal cyclosporine prolongs survival of late-stage ALS mice. Brain Research 2001; 894: 327-331). Animals treated with CsA showed a dramatic 80% survival rate relative to only a 10% survival rate in the absence of treatment. It was later established that this was largely the result of the binding of CsA to mitochondrial CyP-D. It has been subsequently established that the utility of CsA extends to chronic neurodegeneration, as was subsequently demonstrated in a rat model of Lou Gerhig's Disease (ALS) (U.S. Pat. No. 5,972,924) where CsA treatment more than doubled the remaining life-span. It has also recently been shown that CyP-D inactivation in CyP-D knockout mice protects axons in experimental autoimmune encephalomyelitis, an animal model of multiple sclerosis (Forte M et al. Cyclophilin D inactivation protects axons in experimental autoimmune encephalomyelitis, an animal model of multiple sclerosis. PNAS 2007; 104(18): 7558-7563). In an Alzheimer's disease mouse model CyP-D deficiency substantially improves learning and memory and synaptic function (Du H et al. Cyclophilin D deficiency attenuates mitochondrial and neuronal perturbation and ameliorates learning and memory in Alzheimer's disease Nature Medicine 2008, 14(10): 1097-1105). In addition, CsA has been shown to be effective in a rat model of Huntington's (Leventhal L et al. CsA protects striatal neurons in vitro and in vivo from 3-nitropropionic acid toxicity. Journal of Comparative Neurology 2000, 425(4): 471-478), and partially effective in a mouse model of Parkinson's (Matsuura K et al. CsA attenuates degeneration of dopaminergic neurons induced by 6-hydroxydopamine in the mouse brain. Brain Research 1996, 733(1): 101-104). Thus, mitochondrial-dependent necrosis represents a prominent disease mechanism suggesting that inhibition of CyP-D could provide a new pharmacologic treatment strategy for these diseases (Du, 2008). Cellular, Tissue and Organ Injury due to a Loss of Cellular Calcium Ion (Ca^{2÷}) Homeostasis.

Ca²⁺ is involved in a number of physiological processes at a cellular level, including the healthy mitochondrial function. Under certain pathological conditions, such as myocardial infarct, stroke, acute hepatotoxicity, cholestasis, and storage/reperfusion injury of transplant organs, mitochondria lose the ability to regulate calcium levels, and excessive calcium accumulation in the mitochondrial matrix results in the opening of large pores in the inner mitochondrial membrane. (Rasola A. et al. The mitochondrial permeability transition pore and its involvement in cell death and in disease pathogenesis. Apoptosis 2007, 12: 815-833.) Nonselective conductance of ions and molecules up to 1.5 kilodaltons through the pore, a process called mitochondrial permeability transition, leads to swelling of mitochondria and other events which culminate in cell death, including the induction of apoptosis. One of the components of the mitochondrial permeability transition pore (MPTP) is CyP-D. CyP-D is an immunophilin molecule whose isomerase activity regulates opening of the MPTP, and inhibition of the isomerase activity by CsA or CsA analogs inhibits creation of the MPTP, and thus prevents cell death.

### Non-immunosuppressive Cyclosporine Analogue Cyclophilin Inhibitors

Despite the advantageous effects of CsA in the above mentioned indications the concomitant effects of immunosuppression limit the utility of CsA as a CyP inhibitor in clinical practice. At present, there are only a few CsA analogs that have been proven to have little or reduced immunosuppressive activity (i.e. <10% of the immunosppressive potency of CsA) and still retain their ability to bind CyP (i.e. >10% CyP binding capacity as compared to CsA).

NIM 811 (Melle⁴-cyclosporine)

NIM 811 is a fermentation product of the fungus Tolypocladium niveum, modified at amino acid 4 displays no immunosuppressive activity (due to lack of calcineurin binding) yet retains binding affinity for CyP-A (Rosenwirth B A et al. Inhibition of human immunodeficiency virus type 1 replication by SDZ NIM 811, a nonimmunosuppressive Cyclosporine Analogue. Antimicrob Agents Chemother 1994, 38: 1763-1772).

### DEBIO 025 (MeAla³EtVal⁴-Cyclosporin)

DEBIO 025 is a dual chemical modification of CsA at amino acids 3 and 4, also displays no immunosuppressive activity yet retains binding affinity for CyP-A PPlase activity (Kristal, 2004).

SCY-635 (DimethylaminoethylthioSar³-hydroxyLeu⁴-Cyclosporin)

SCY-635 is a dual chemical modification of CsA at amino acids 3 and 4, also displays no immunosuppressive activity yet retains binding affinity for CyP-A PPlase activity (PCT Publication No. WO2006/039668).

Generally, these compounds have modification on the face of CsA that is responsible for binding calcineurin, and generally require the modification of amino acids 3 and 4. The modification of amino acids 3 and 4 is a laborious and complex, as this approach typically involves opening up the cyclosporine ring, replacing and/or modifying those amino acids and then closing up the ring to produce the modified cyclosporine.

In contrast, modification of the side chain of amino acid 1 does not require opening of the cyclosporine ring. However, amino acid 1 is associated with CyP binding (as opposed to calcineurin binding) and has been modified to increase the immunosuppressive efficacy of CsA. For example U.S. Pat. No. 6,605,593, discloses a single modification of amino acid 1 that results in a CsA analog with increased immunosuppressive potency.

Therefore, it would be desirable to have a non-immunosuppressive Cyclosporine analogue molecule (a “NICAM”) that are readily synthesized and are efficacious in the treatment of CyP mediated diseases.

### SUMMARY OF THE INVENTION

One aspect of the invention relates to compounds represented by the chemical structure of Formula I:

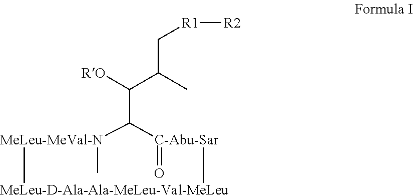

wherein
   a. R′ is H or Acetyl;
   b. R1 is a saturated or unsaturated straight chain or branched aliphatic carbon chain from 2 to 15 carbon atoms in length; and
   c. R2 is selected from the group consisting of:
      (i) a H;
      (ii) an unsubstituted, N-substituted, or N,N-disubstituted amide;
      (iii) a N-substituted or unsubstituted acyl protected amine;
      (iv) a carboxylic acid;
      (v) a N-substituted or unsubstituted amine;
      (vi) a nitrile;
      (vii) an ester;
      (viii) a ketone;
      (ix) a hydroxy, dihydroxy, trihydroxy, or polyhydroxy alkyl; and
      (x) a substituted or unsubstituted aryl;
         or a pharmaceutically acceptable salt thereof.

A second aspect of the invention relates to compounds of Formula II:

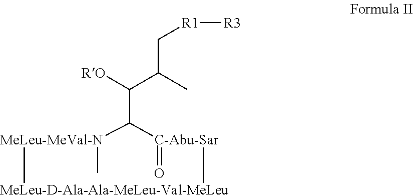

wherein
a. R′ is H or Acetyl;
b. R1 is a saturated or unsaturated straight chain or branched aliphatic carbon chain from 2 to 15 carbon atoms in length; and
c. R3 is selected from the group consisting of
   (i) a saturated or unsaturated straight or branched aliphatic carbon chain containing one or more substituents selected from the group consisting of a hydrogen, a ketone, a hydroxyl, a nitrile, a carboxylic acid, an ester and a 1,3-dioxolane;
   (ii) an aromatic group containing one or more substituents selected from the group consisting of a halide, an ester and nitro; and
   (iii) a combination of said saturated or unsaturated, straight or branched aliphatic chain and said aromatic group.
      or a pharmaceutically acceptable salt thereof.

A third aspect of the invention relates to compounds of Formula III:

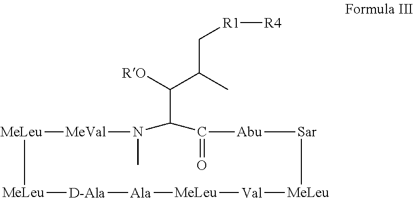

wherein
a. R′ is H or Acetyl;
b. R1 is a saturated or unsaturated straight chain or branched aliphatic carbon chain from 2 to 15 carbon atoms in length; and
c. R4 is selected from the group consisting of

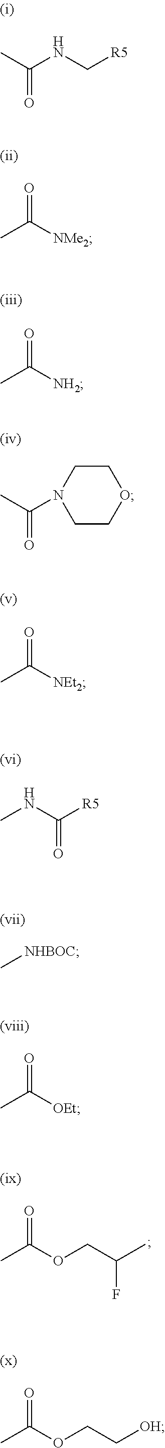

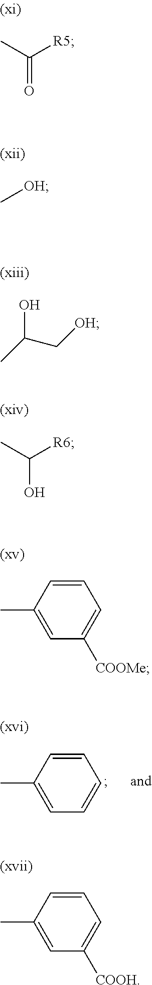

wherein
   1. R5 is a saturated or unsaturated straight chain or branched aliphatic carbon chain between 1 and 10 carbons in length
   2. R6 is a monohydroxylated, dihydroxylated, trihydroxylated or polyhydroxylated saturated or unsaturated straight chain or branched aliphatic carbon chain between 1 and 10 carbons in length;
      or a pharmaceutically acceptable salt thereof.

A fourth aspect of the invention relates to compounds Formula IV:

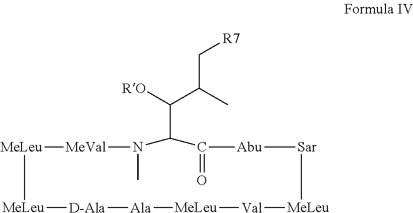

wherein
   I. R′ is H or Acetyl; and
   II. R7 is selected from the group consisting of:

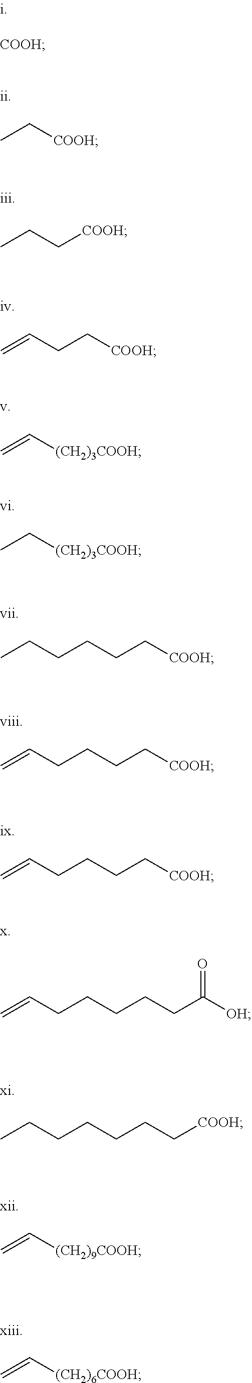

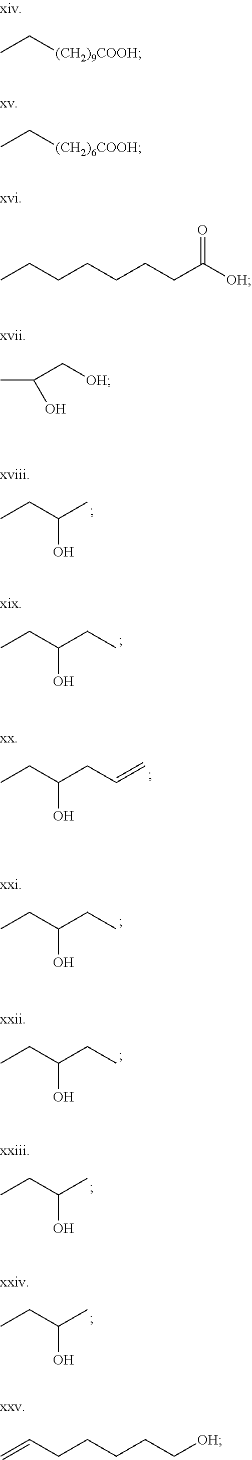

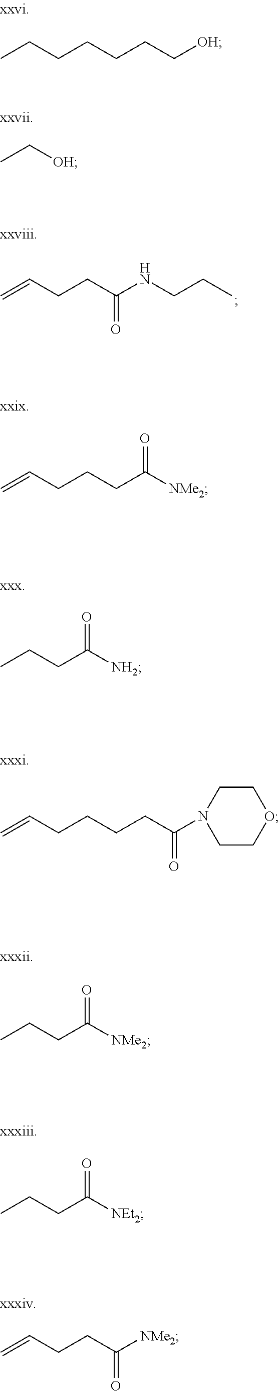

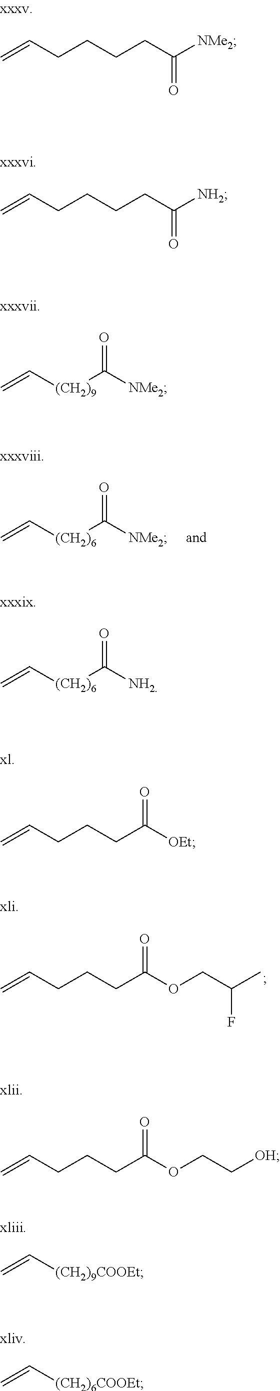

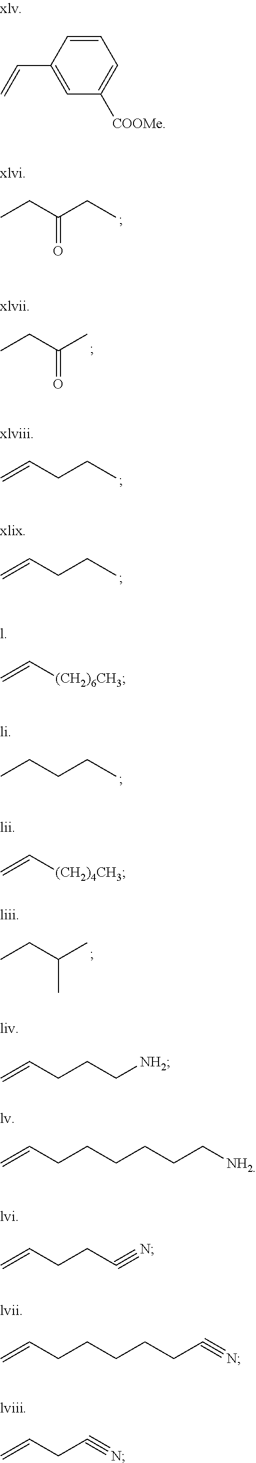

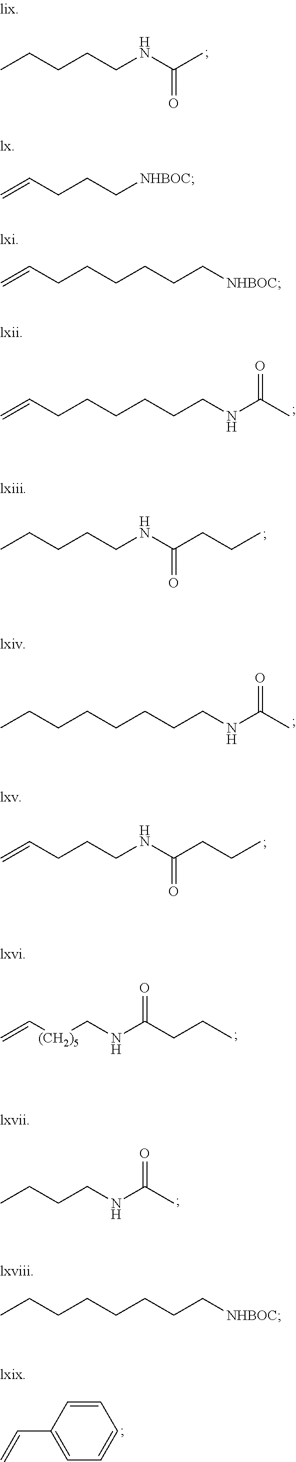

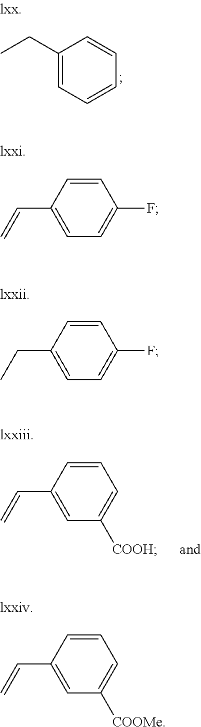

or a pharmaceutically acceptable salt thereof.

According to another aspect of the invention, there is provided a process to produce a compound of said Formula I, comprising the steps of
a. reacting acetyl CsA aldehyde modified at amino acid 1 of Formula IX:

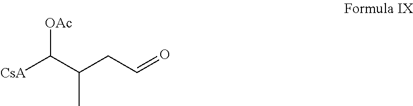

with a phosphonium salt of Formula VIII:

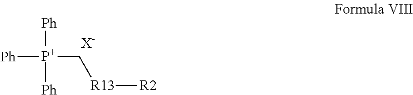

wherein
   I. R13 is a saturated or unsaturated straight chain or branched aliphatic carbon chain from 1 to 14 carbon atoms in length; and
   II. R2 is as defined above for Formula I;
in the presence of a base to produce an acetylated compound of Formula X:

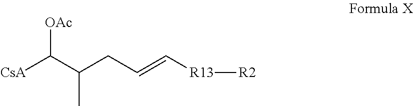

b. deacetylating the compound of Formula X using a base; and
c. where R1 is saturated, hydrogenating the double bond of the compound of Formula X by reacting the compound with a hydrogenating agent to produce a saturated analogue of Formula I.

According to another aspect of the invention, there is provided a process to produce a non-immunosuppressive compound of Formula XIV:

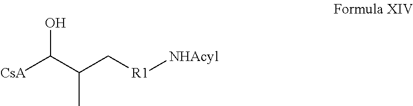

comprising the steps of
a. reacting the compound of Formula XV:

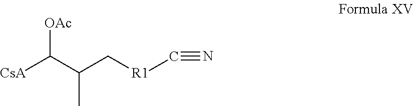

in the presence of a reducing agent and an acylating agent to produce acetylated compounds of Formula XVI:

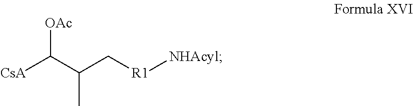

and
b. deacetylating the compound of Formula XVI using a base;
wherein R1 of Formulae XIV, XV and XVI is a saturated or unsaturated straight chain or branched aliphatic carbon chain between 2 and 15 carbons in length.

According to another aspect of the invention, there is provided a process to produce a non-immunosuppressive compound of Formula XXI:

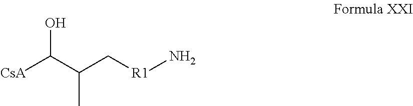

comprising the steps of
a. by dissolving the compound of Formula XX:

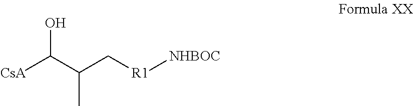

in an anhydrous solvent; and
b. reacting the solution with trifluoroacetic acid (TFA);
wherein R1 of Formulae XX and XXI is a saturated or unsaturated, straight chain or branched aliphatic carbon chain between 2 and 15 carbons in length.

According to another aspect of the invention, there is provided a process to produce a non-immunosuppressive compound of Formula XIV:

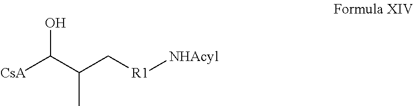

comprising the steps of
a. dissolving the compound of Formula XXI:

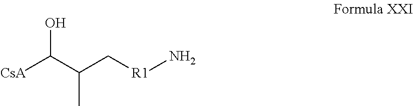

in anhydrous pyridine;
b. reacting the solution with acylating agent; and
c. removing the solvent to yield the compound of Formula XIV;
wherein R1 of Formulae XIV and XXI is a saturated or unsaturated straight chain or branched aliphatic carbon chain between 2 and 15 carbons in length.

According to another aspect of the invention, there is provided a process to produce a non-immunosuppressive compound of Formula XXIV:

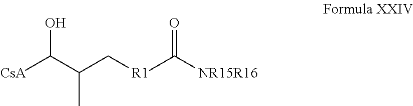

wherein
   I. R1 is a saturated or unsaturated, straight or branched aliphatic carbon chain between 2 and 15 carbons in length; and
   II. R15 and R16 are independently hydrogen or a saturated or unsaturated straight chain or branched aliphatic group; or where NR15R16 together forms a morpholinyl moiety;
comprising the steps of
a. by combining the compound of Formula XXV:

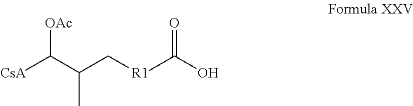

with thionylchloride to yield a residue of the Formula XXVI;

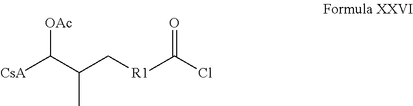

b. dissolving the residue in anhydrous solvent and reacting with a compound of the Formula XXVII:

R15R16NH Formula XXVII
to yield the compound of Formula XXVIII

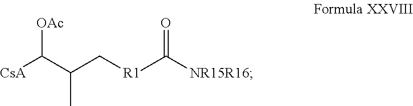

and
c. deacetylating the compound of Formula XXIV with a base.

According to another aspect of the invention, there is provided a process to produce a non-immunosuppressive compound of Formula XXIV:

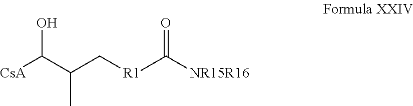

wherein
   I. R1 is a saturated or unsaturated straight chain or branched aliphatic carbon chain between 2 and 15 carbons in length;
   II. R15 and R16 are independently hydrogen or a saturated or unsaturated straight chain or branched aliphatic group; or where NR15R16 together forms a morpholinyl moiety;
comprising the steps of
a. dissolving the compound of Formula XXV:

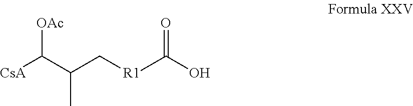

in anhydrous solvent under nitrogen;
b. reacting with dicyclohexylcarvodiimide, 1-hydroxybenzotriazole hydrate and the compound of the Formula XVIII;

R15R16NH Formula XXVII
and
c. deacetylating the compound of Formula XVIII with a base.

According to another aspect of the invention, there is provided a process to produce a non-immunosuppressive compound of Formula XXXII:

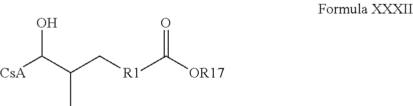

wherein
   I. R1 is a saturated or unsaturated straight chain or branched aliphatic carbon chain between 2 and 15 carbons in length; and
   II. R17 is a saturated or unsaturated straight chain or branched aliphatic group, optionally containing a halogen or hydroxyl substituent;
by reacting the compound of Formula XXX:

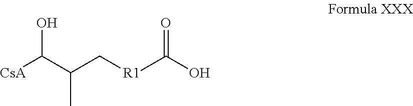

with a compound of Formula XXXI:

R17OH Formula XXXI
in the presence of an acid.

According to another aspect of the invention, there is provided a process to produce a non-immunosuppressive compound of Formula XXVI:

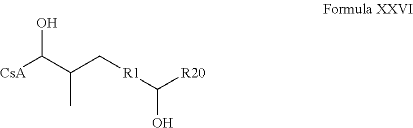

wherein
   I. R1 is a saturated or unsaturated straight chain or branched aliphatic carbon chain between 2 and 15 carbons in length; and
   II. R20 is a saturated or unsaturated straight chain or branched aliphatic group;
by reacting the compound of Formula XXXV:

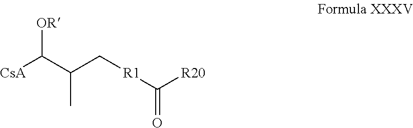

wherein R′ is optionally H or acetyl
with sodium borohydride; and
where R′ is acetyl, deacetylating the compound of Formula XXXV with a base.

According to another aspect of the invention, there is provided a process to produce a non-immunosuppressive compound of Formula XXIX:

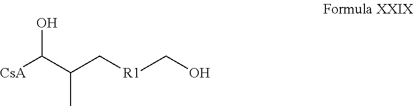

wherein R1 is a saturated or unsaturated straight chain or branched aliphatic carbon chain between 2 and 15 carbons in length;
by reacting the compound of Formula XXVIII:

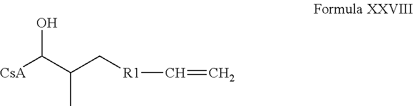

with borane-tetrahydrofuran and sodium peroxide.

According to another aspect of the invention, there is provided a process to produce a non-immunosuppressive compound of Formula XLIII:

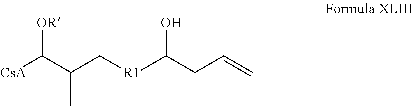

wherein
   I. R′ is H or Acetyl; and
   II. R1 is a saturated or unsaturated, straight or branched aliphatic chain from 2 to 15 carbons in length;
by reacting the compound of Formula XLI:

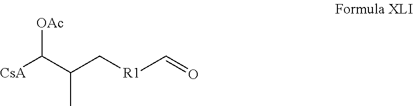

with the compound of Formula XLII;

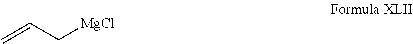

in an anhydrous solvent; and
deacetylating the compound of Formula XVIII with a base.

According to another aspect of the invention, there is provided a process to produce a non-immunosuppressive compound of Formula XLVI:

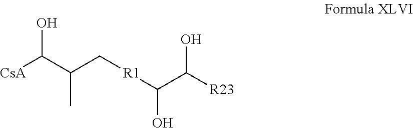

wherein
   I. R1 is a saturated or unsaturated straight chain or branched aliphatic carbon chain from 2 to 15 carbon atoms in length; and
   II. R23 is a saturated or unsaturated straight chain or branched aliphatic group;
comprising the steps of:
   a. reacting the compound of Formula XLV

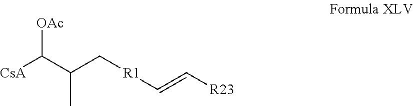

with hydrogen peroxide and formic acid;
b. reacting the product with a base to yield the compound of Formula XLVI; and
c. deacetylating the compound of Formula XLV with a base.

The present invention discloses non-immunosuppressive cyclosporine analogues. Such compounds bind CyP and are potentially useful in treating CyP mediated diseases.

In general, for Formulae I through XLVI:

“Carboxylic acid” includes a group in which the carboxylic acid moiety is connected to one of the following substituents:
1. alkyl which may be substituted (for example, alkyl of 2 to 15 carbons);
2. alkenyl which may be substituted (for example, alkenyl of 2 to 15 carbons); and
3. alkynyl which may be substituted (for example, alkynyl of 2 to 15 carbons);

The substituents of the above-described above may include halogen (for example, fluorine, chlorine, bromine, iodine, etc.), nitro, cyano, hydroxy, thiol which may be substituted (for example, thiol, C1-4 alkylthio, etc.), amino which may be substituted (for example, amino, mono-C1-4 alkylamino, di-C1-4 alkylamino, 5- to 6-membered cyclic amino such as tetrahydropyrrole, piperazine, piperidine, morpholine, thiomorpholine, pyrrole, imidazole, etc.), C1-4 alkoxy which may be halogenated (for example, methoxy, ethoxy, propoxy, butoxy, trifluoromethoxy, trifluoroethoxy, etc.), C1-4 alkoxy-C1-4 alkoxy which may be halogenated (for example, methoxymethoxy, methoxyethoxy, ethoxyethoxy, trifluoromethoxyethoxy, trifluoroethoxyethoxy, etc.), formyl, C2-4 alkanoyl (for example, acetyl, propionyl, etc.), C1-4 alkylsulfonyl (for example, methanesulfonyl, ethanesulfonyl, etc.), and the like, and the number of the substituents is preferably 1 to 3.

Further, the substituents of the above “amino which may be substituted” may bind each other to form a cyclic amino group (for example, a group which is formed by subtracting a hydrogen atom from the ring constituting nitrogen atom of a 5- to 6-membered ring such as tetrahydropyrrole, piperazine, piperidine, morpholine, thiomorpholine, pyrrole, imidazole, etc. so that a substituent can be attached to the nitrogen atom, or the like). The cyclic amino group may be substituted and examples of the substituent include halogen (for example, fluorine, chlorine, bromine, iodine, etc.), nitro, cyano, hydroxy, thiol which may be substituted (for example, thiol, C1-4 alkylthio, etc.), amino which may be substituted (for example, amino, mono-C.sub.1-4 alkylamino, di-C1-4 alkylamino, 5- to 6-membered cyclic amino such as tetrahydropyrrole, piperazine, piperidine, morpholine, thiomorpholine, pyrrole, imidazole, etc.), carboxyl which may be esterified or amidated (for example, carboxyl, C1-4 alkoxy-carbonyl, carbamoyl, mono-C1-4 alkyl-carbamoyl, di-C1-4 alkyl-carbamoyl, etc.), C1-4 alkoxy which may be halogenated (for example, methoxy, ethoxy, propoxy, butoxy, trifluoromethoxy, trifluoroethoxy, etc.), C1-4 alkoxy-C.sub.1-4 alkoxy which may halogenated (for example, methoxymethoxy, methoxyethoxy, ethoxyethoxy, trifluoromethoxyethoxy, trifluoroethoxyethoxy, etc.), formyl, C2-4 alkanoyl (for example, acetyl, propionyl, etc.), C1-4 alkylsulfonyl (for example, methanesulfonyl, ethanesulfonyl), and the like, and the number of the substituents is preferably 1 to 3. “Amine” includes a group which may be unsubstituted or in which the amine moiety is an N-substituted or N,N disubstituted having one or two substituents which may be independently selected from:
1. alkyl which may be substituted (for example, alkyl of 2 to 15 carbons);
2. alkenyl which may be substituted (for example, alkenyl of 2 to 15 carbons);
3. alkynyl which may be substituted (for example, alkynyl of 2 to 15 carbons);
4. formyl or acyl which may be substituted (for example, alkanoyl of 2 to 4 carbons (for example, acetyl, propionyl, butyryl, isobutyryl, etc.), alkylsulfonyl of 1 to 4 carbons (for example, methanesulfonyl, ethanesulfonyl, etc.) and the like);
5. aryl which may be substituted (for example, phenyl, naphthyl, etc.); and the like;
   and connected to a substituent independently selected from the substituents as defined for “carboxylic acid” above.

“Amide” includes a compound in which the carboxylic group of the amide moiety is connected to a substituent independently selected from the substituents as defined for “carboxylic acid” above, connect to the amino group of the amide moiety is an N-substituted or N,N disubstituted having one or two substituents, respectively, which may be independently selected from:
1. alkyl which may be substituted (for example, alkyl of 2 to 15 carbons);
2. alkenyl which may be substituted (for example, alkenyl of 2 to 15 carbons);
3. alkynyl which may be substituted (for example, alkynyl of 2 to 15 carbons);
4. formyl or acyl which may be substituted (for example, alkanoyl of 2 to 4 carbons (for example, acetyl, propionyl, butyryl, isobutyryl, etc.), alkylsulfonyl of 1 to 4 carbons (for example, methanesulfonyl, ethanesulfonyl, etc.) and the like);
5. aryl which may be substituted (for example, phenyl, naphthyl, etc.); and the like

“Aryl” may be exemplified by a monocyclic or fused polycyclic aromatic hydrocarbon group, and for example, a C6-14 aryl group such as phenyl, naphthyl, anthryl, phenanthryl or acenaphthylenyl, and the like are preferred, with phenyl being preferred. Said aryl may be substituted with one or more substitutuents, such as lower alkoxy (e.g., C1-6 alkoxy such as methoxy, ethoxy or propoxy, etc.), a halogen atom (e.g., fluorine, chlorine, bromine, iodine, etc.), lower alkyl (e.g., C1-6 alkyl such as methyl, ethyl or propyl, etc.), lower alkenyl (e.g., C2-6 alkenyl such as vinyl or allyl, etc.), lower alkynyl (e.g., C.2-6 alkynyl such as ethynyl or propargyl, etc.), amino which may be substituted, hydroxyl which may be substituted, cyano, amidino which may be substituted, carboxyl, lower alkoxycarbonyl (e.g., C1-6 alkoxycarbonyl such as methoxycarbonyl or ethoxycarbonyl, etc.), carbamoyl which may be substituted (e.g., carbamoyl which may be substituted with C1-6 alkyl or acyl (e.g., formyl, C2-6 alkanoyl, benzoyl, C1-6 alkoxycarbonyl which may be halogenated, C1-6 alkylsulfonyl which may be halogenated, benzenesulfonyl, etc.) which may be substituted with a 5- to 6-membered aromatic monocyclic heterocyclic group (e.g., pyridinyl, etc.), 1-azetidinylcarbonyl, 1-pyrrolidinylcarbonyl, piperidinocarbonyl, morpholinocarbonyl, thiomorpholinocarbonyl (the sulfur atom may be oxidized), 1-piperazinylcarbonyl, etc.), or the like. Any of these substituents may be independently substituted at 1 to 3 substitutable positions.

“Ketone” includes a compound in which the carbonyl group of the ketone moiety is connected to one or two substituents independently selected from the substituents as defined above for said “carboxylic acid”.

“Ester” includes either a carboxylic or an alcohol ester wherein of the ester group is composed of one or two substituents independently selected from the substituents as defined for “carboxylic acid” or “aryl”.

“Alkyl” unless otherwise defined is preferably an alkyl of 1 to 15 carbon units in length.

“Aromatic group” may be exemplified by aryl as defined above, or a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl or the like; and a 8- to 16-membered (preferably, 10- to 12-membered) aromatic fused heterocyclic group.

“Non-immunosuppresive” refers to the ability of a compound to exhibit a substantially reduced level of suppression of the immune system as compared with CsA, as measured by the compounds ability to inhibit the proliferation of human lymphocytes in cell culture and preferably as measured by the method set out in Example 19 below.

“Analogue” means a structural analogue of CsA which differs from CsA in one or more functional groups. Preferably, such analogues preserve at least a substantial portion of the ability of CsA to bind CyP.

Preferred species of Formula I are those in which R′ is H, R1 is a saturated or unsaturated alkyl between 2 and 15 carbons in length and R2 is selected from:
1. carboxylic acid comprising a carboxyl group;
2. N-substituted of N,N-disubstituted amide wherein the substituents are independently selected from an H, an alkyl of between 1 and 7 carbons in length, or said substituents form a heterocylic ring of which the heterocyle is selected from O, N or S;
3. an ester of between 1 and 7 carbons in length;
4. an monohydroxylated, or dihydroxylated alkyl of between 1 and 7 carbons in length;
5. a N-substituted or unsubstituted acyl protected amine of between 1 and 7 carbons in length;
6. a nitrile;
7. a ketone wherein the carboxylic group of the ketone is connected to R1 and saturated or unsaturated alkyl chain of between 1 and 7 carbons in length;
8. phenyl, optionally substituted with one or more substituents independently selected from nitrogen dioxide, a fluorine, an amine, an ester or a carboxyl group.

The compounds of the present invention may exist in the form of optically active compounds. The present invention contemplates all enantiomers of optically active compounds within the scope of the above formulae, both individually and in mixtures of racemates. As well, the present invention includes prodrugs of the compounds defined herein.

According to another aspect, compounds of the present invention may be useful for treating or preventing or studying a cyclophilin mediated disease in a mammal, preferably a human. Such disease is usually mediated by the over expression of cyclophilin, such as a congenital over expression of cyclophillin.

Cyclophilin mediated diseases which may be treated by compounds of the present invention include:
a. a viral infection;
b. inflammatory disease;
c. cancer;
d. muscular degenerative disorder;
e. neurodegenerative disorder; and
f. injury associated with loss of cellular calcium homeostasis.

Said viral infection may be caused by a virus selected from the group consisting of Human Immunodeficiency virus, Hepatitis A, Hepatitis B, Hepatitis C, Hepatitis D, and Hepatitis E. Said inflammatory disease is selected from the group consisting of asthma, autoimmune disease, chronic inflammation, chronic prostatitis, glomerulonephritis, hypersensitivity disease, inflammatory bowel disease, sepsis, vascular smooth muscle cell disease, aneurysms, pelvic inflammatory disease, reperfusion injury, rheumatoid arthritis, transplant rejection, and vasculitis. Said cancer may be selected from the group consisting of small and non-small cell lung, bladder, hepatocellular, pancreatic and breast cancer. Said muscular degenerative disorder may selected from the group consisting of myocardial reperfusion injury, muscular dystrophy, and collagen VI myopathies. Said neurodegenerative disorder may be selected from the group consisting of Alzheimer's disease, Parkinson's disease, Huntington's disease, Multiple Systems Atrophy, Multiple Sclerosis, cerebral palsy, stroke, diabetic neuropathy, amyotrophic lateral sclerosis (Lou Gehrig's Disease), spinal cord injury, and cerebral injury. Said injury associated with loss of cellular calcium homeostasis may be selected from the group consisting of myocardial infarct, stroke, acute hepatotoxicity, cholestasis, and storage/reperfusion injury of transplant organs.

### BRIEF DESCRIPTION OF THE DRAWINGS

These and other advantages of the invention will become apparent upon reading the following detailed description and upon referring to the drawings in which:

FIG. 1 is a line graph depicting the inhibition of CyP-D as measured by mitochondrial absorbance following addition of calcium chloride in the absence or presence of a CsA.

### DETAILED DESCRIPTION

The compounds of this invention may be administered neat or with a pharmaceutical carrier to a warm-blooded animal in need thereof. The pharmaceutical carrier may be solid or liquid. The inventive mixture may be administered orally, topically, parenterally, by inhalation spray or rectally in dosage unit formulations containing conventional non-toxic pharmaceutically acceptable carriers, adjuvants and vehicles. The term parenteral, as used herein, includes subcutaneous injections, intravenous, intramuscular, intrasternal injection or infusion techniques.

The pharmaceutical compositions containing the inventive mixture may preferably be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. Tablets containing the active ingredient in admixture with non-toxic pharmaceutically acceptable excipients may also be manufactured by known methods. The excipients used may be for example, (1) inert diluents such as calcium carbonate, lactose, calcium phosphate or sodium phosphate; (2) granulating and disintegrating agents such as corn starch, or alginic acid; (3) binding agents such as starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,160,452; and 4,265,874 to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example peanut oil, liquid paraffin, or olive oil.

Aqueous suspensions normally contain the active materials in admixture with excipients suitable for the manufacture of aqueous suspensions. Such excipients may include: (1) suspending agents such as sodium carboxymethylcellulose, methylcellulose, hydroxypropylmethylcellulose, sodium alginate, polyvinylpyrrolidone, gum tragacanth and gum acacia; or (2) dispersing or wetting agents which may be a naturally-occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived from a fatty acid and a hexitol such as polyoxyethylene sorbitol monooleate, or a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents such as sucrose, aspartame or saccharin.

Oily suspension may be formulated by suspending the active ingredient in a vegetable oil, for example arachis oil, olive oil, sesame oil or coconut oil, a fish oil which contains omega 3 fatty acid, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an antioxidant such as ascorbic acid.

Dispersible powders and granules are suitable for the preparation of an aqueous suspension. They provide the active ingredient in a mixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions containing the inventive mixture may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soy bean and lecithin, (3) esters or partial ester 30 derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Syrups and elixirs may be formulated with sweetening agents, for example, glycerol, propylene glycol, sorbitol, aspartame or sucrose. Such formulations may also contain a demulcent, a preservative, and flavoring and coloring agents.

The pharmaceutical compositions may be in the form of a sterile injectable aqueous or oleagenous suspension. This suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The inventive mixture may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, suitable creams, ointments, jellies, solutions or suspensions, etc., containing that normally are used with cyclosporine may be employed.

In a particularly preferred embodiment, a liquid solution containing a surfactant, ethanol, a lipophilic and/or an amphiphilic solvent as non-active ingredients is used. Specifically, an oral multiple emulsion formula containing the isomeric analogue mixture and the following non-medicinal ingredients: d-alpha Tocopheryl polyethylene glycol 1000 succinate (vitamin E TPGS), medium chain triglyceride (MCT) oil, Tween 40, and ethanol is used. A soft gelatin capsule (comprising gelatin, glycerin, water, and sorbitol) containing the compound and the same non-medicinal ingredients as the oral solution may also preferably be used.

It will be understood, however, that the specific dose level for any particular patient will depend upon a variety of factors including the activity of the specific compound employed, the age, body weight, general health, sex, diet, time of administration, route of administration, rate of excretion, drug combination and the nature and severity of the particular disease or condition undergoing therapy.

### Methodology

Reactions 1 to 18, set out below, are general examples of the chemical reactions able to synthesize the desired compounds modified at amino acid 1 of CsA; hereinafter depicted as

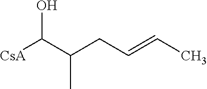

using reagents that have the requisite chemical properties, and it would be understood by a person skilled in the art that substitutions of certain reactants may be made.

The identity and purity of the prepared compounds were generally established by methodologies including mass spectrometry, HPLC and NMR spectroscopy. Mass spectra (ESI-MS) were measured on a Hewlett Packard 1100 MSD system. NMR spectra were measured on a Varian MercuryPlus 400 MHz spectrometer in deuterated solvents (DMSO for phosphonium salts, benzene for all other compounds). Analytical and preparative reversed phase HPLC was carried out on an Agilent 1100 Series system.

### Synthesis of Phosphonium Salt Compounds

Phosphonium salts are prepared through reaction of triphenylphosphine or any other suitable phosphines with alkyl halides (R—X; X═Cl, Br, or I). Suitable alkyl halides are any primary or any secondary aliphatic halide of any chain length or molecular weight. These alkyl halides may be branched or unbranched, saturated or unsaturated.

If the reaction is carried out in toluene (Reaction 1), the product precipitates directly from the reaction solution. Unreactive substrates, however, require a more polar solvent such as dimethylformamide (DMF) (Reaction 2) to shorten reaction times and to achieve satisfactory yields.

### Reaction 1

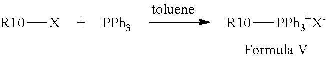

Where X is a halide (including but not limited to Cl, Br, and I), and R10 is a saturated or unsaturated. straight or branched aliphatic chain, optionally containing a substituent selected from the group of ketones, hydroxyls, nitriles, carboxylic acids, esters and 1,3-dioxolanes; an aromatic group, optionally containing a substituent selected from the group of halides, esters and nitro; or a combination of the aforementioned saturated or unsaturated, straight or branched aliphatic chain and the aformentioned aromatic groups.

### Example 1

### Synthesis of 404-15

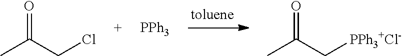

As an illustrative example, triphenylphosphine (13 mmol) is dissolved in 50 mL toluene and chloroacetone (10 mmol) is added to give a clear solution. The reaction is stirred under reflux over night. A colorless solid is filtered off, washed with toluene and hexane and dried in vacuum.

Using Reaction 1, the following compounds are further examples of the compounds that may be synthesized.

| Compound | Reactant (R10-X) | Conditions |
|---|---|---|
| 404-08 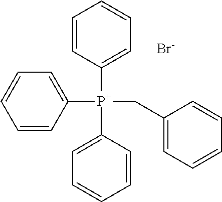 [SMILES: c1ccc(C[P+](c2ccccc2)(c2ccccc2)c2ccccc2)cc1 ; [Br-]] | benzyl bromide | 4 hours at reflux |
| 404-09 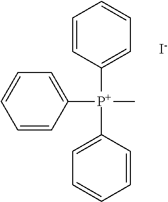 [SMILES: C[P+](c1ccccc1)(c1ccccc1)c1ccccc1 ; [I-]] | methyl iodide | RT over night |
| 404-12 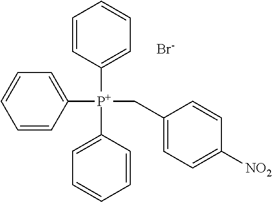 [SMILES: O=[N+]([O-])c1ccc(C[P+](c2ccccc2)(c2ccccc2)c2ccccc2)cc1 ; [Br-]] | 4-nitrobenzyl bromide | 6 hours at reflux |
| 404-15 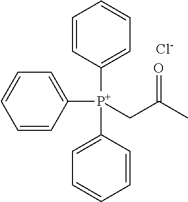 [SMILES: CC(=O)C[P+](c1ccccc1)(c1ccccc1)c1ccccc1 ; [Cl-]] | chloracetone | reflux over night |
| 404-64 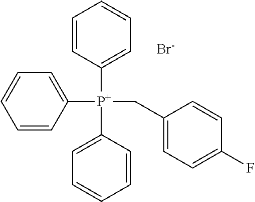 [SMILES: Fc1ccc(C[P+](c2ccccc2)(c2ccccc2)c2ccccc2)cc1 ; [Br-]] | 4-fluorobenzyl bromide | reflux over night |
| 404-77 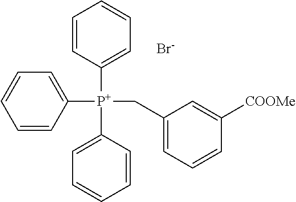 [SMILES: COC(=O)c1cccc(C[P+](c2ccccc2)(c2ccccc2)c2ccccc2)c1 ; [Br-]] | methyl 3- bromomethylbenzoate | 6 hours at reflux |
| 404-87 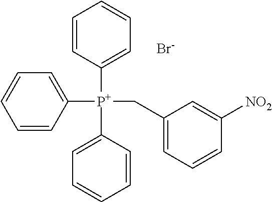 [SMILES: O=[N+]([O-])c1cccc(C[P+](c2ccccc2)(c2ccccc2)c2ccccc2)c1 ; [Br-]] | 3-nitrobenzyl bromide | 6 hours at reflux |
| 404-161 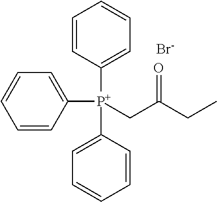 [SMILES: CCC(=O)C[P+](c1ccccc1)(c1ccccc1)c1ccccc1 ; [Br-]] | 1-bromo-2-butanone | RT over night |
| 404-170 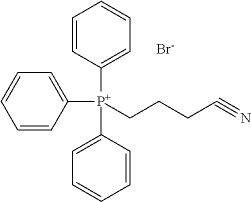 [SMILES: N#CCCC[P+](c1ccccc1)(c1ccccc1)c1ccccc1 ; [Br-]] | 4-bromobutyronitrile | reflux over night |

Alternatively, suitable phosphonium salts may be synthesized through Reaction 2 as illustrated below:

### Reaction 2

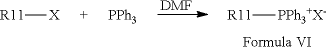

Where X is a halide (including but not limited to Cl, Br, and I), and R10 is a saturated or unsaturated. straight or branched aliphatic chain, optionally containing a substituent selected from the group of ketones, hydroxyls, nitriles, carboxylic acids, esters and 1,3-dioxolanes; an aromatic group, optionally containing a substituent selected from the group of halides, esters and nitro; or a combination of the aforementioned saturated or unsaturated, straight or branched aliphatic chain and the aformentioned aromatic groups.

### Example 2

### Synthesis of 404-51

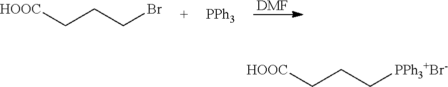

As an illustrative example, triphenylphosphine (11 mmol) is dissolved in 10 mL DMF and 4-bromobutyric acid (10 mmol) is added. The reaction is stirred for 7 hours at 110° C. and is then allowed to cool over night. Fifty mL toluene is added and a crystalline, colorless solid is collected by filtration. The product is washed with toluene and hexane and dried in vacuum over night.

If crystallization does not set in after treatment with toluene, the product is extracted with 20 mL MeOH/H₂O (1:1 mixture). The aqueous phase is washed with toluene and hexane and brought to dryness. The residue is stirred with 50 mL ethyl acetate (EtOAc) at reflux temperature for 20-30 min. If a crystalline solid is obtained, the product is collected by filtration, washed with EtOAc and hexane and dried. In case the product is obtained as an oil or gum, the EtOAc is decanted and the remaining product is dried in vacuum.

Using Reaction 2, the following compounds are further examples of the compounds that may be synthesized.

| Compound | Reactant (R11-X) | Conditions |
|---|---|---|
| 404-14 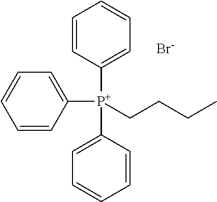 [SMILES: CCCC[P+](c1ccccc1)(c1ccccc1)c1ccccc1 ; [Br-]] | 1-bromobutane | 6.5 hours at 120° C. |
| 404-29 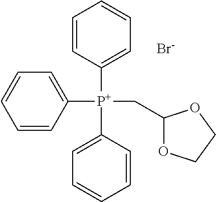 [SMILES: c1ccc([P+](CC2OCCO2)(c2ccccc2)c2ccccc2)cc1 ; [Br-]] | 2-bromomethyl-1,3- dioxolane | 120° C. over night |
| 404-34 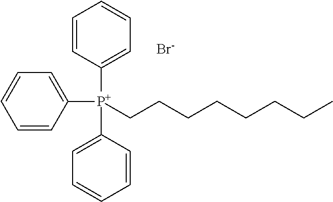 [SMILES: CCCCCCCC[P+](c1ccccc1)(c1ccccc1)c1ccccc1 ; [Br-]] | 1-bromooctane | 110° C. over night |
| 404-51 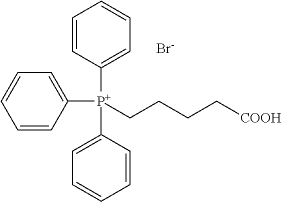 [SMILES: O=C(O)CCCC[P+](c1ccccc1)(c1ccccc1)c1ccccc1 ; [Br-]] | 5-bromovaleric acid | 8 hours at 120° C. |
| 404-78 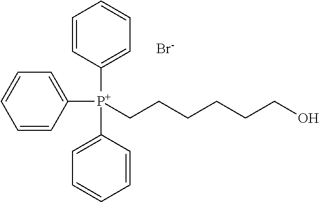 [SMILES: OCCCCCC[P+](c1ccccc1)(c1ccccc1)c1ccccc1 ; [Br-]] | 6-bromohexanol | 110° C. over night |
| 404-116 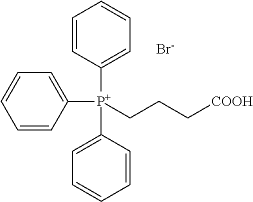 [SMILES: O=C(O)CCC[P+](c1ccccc1)(c1ccccc1)c1ccccc1 ; [Br-]] | 4-bromobutyric acid | 7 hours at 110° C. |
| 416-01 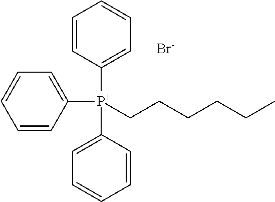 [SMILES: CCCCCC[P+](c1ccccc1)(c1ccccc1)c1ccccc1 ; [Br-]] | 1-bromohexane | 110° C. over night |
| 416-02 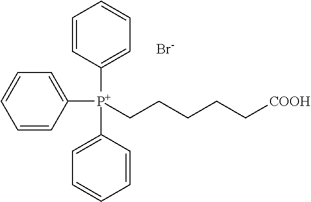 [SMILES: O=C(O)CCCCC[P+](c1ccccc1)(c1ccccc1)c1ccccc1 ; [Br-]] | 6-bromohexanoic acid | 110° C. over night |
| 419-132 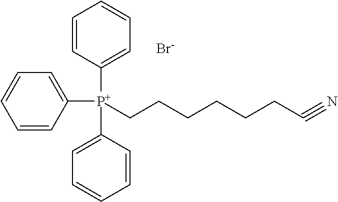 [SMILES: N#CCCCCCC[P+](c1ccccc1)(c1ccccc1)c1ccccc1 ; [Br-]] | 7-bromoheptanenitrile | 110° C. over night |
| 419-134 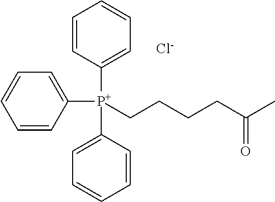 [SMILES: CC(=O)CCCC[P+](c1ccccc1)(c1ccccc1)c1ccccc1 ; [Cl-]] | 6-chloro-2-hexanone | 110° C. over night |
| 419-136 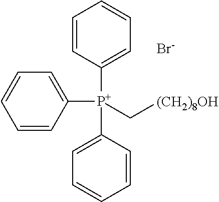 | 9-bromo-1-nonanol | 110° C. over night |
| 420-32 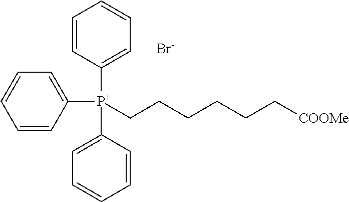 [SMILES: COC(=O)CCCCCC[P+](c1ccccc1)(c1ccccc1)c1ccccc1 ; [Br-]] | methyl 7-bromohexanoate | 110° C. over night |
| 420-78 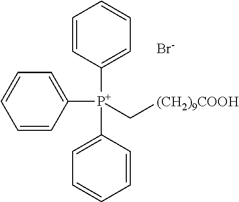 | 11-bromoundecanoic acid | 110° C. over night |
| 420-80 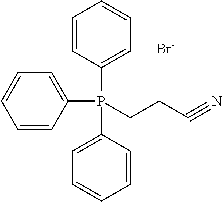 [SMILES: N#CCC[P+](c1ccccc1)(c1ccccc1)c1ccccc1 ; [Br-]] | 3-bromopropionitrile | 110° C. over night |
| 420-82 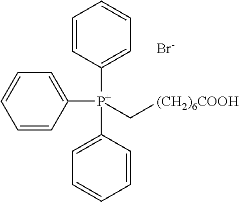 | 8-bromooctanoic acid | 110° C. over night |
| 420-90 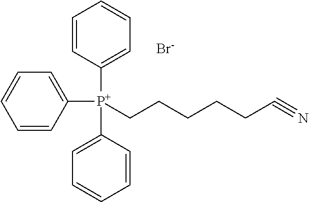 [SMILES: N#CCCCCC[P+](c1ccccc1)(c1ccccc1)c1ccccc1 ; [Br-]] | 6-bromohexanenitrile | 110° C. over night |
| 420-94 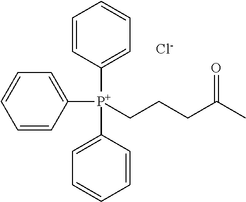 [SMILES: CC(=O)CCC[P+](c1ccccc1)(c1ccccc1)c1ccccc1 ; [Cl-]] | 5-chloro-2-pentanone | 110° C. over night |

### Wittig Reaction

The Wittig reaction is broadly applicable to a wide range of substrates and reactants. The side chain, which is introduced to the substrate in the reaction, can represent any number of branched and unbranched, saturated and unsaturated aliphatic compounds of variable length (R′) and may contain a broad range of functional groups.

In the Wittig reaction, a base, such as potassium tert-butoxide (KOtBu) is used to generate an ylide from a phosphonium salt. The ylide reacts with the carbonyl group of the substrate, CsA-aldehyde, to form an alkene. Phosphonium salts containing a carboxylic acid side chain require at least two equivalents of base to generate the ylide.

### Reaction 3

### Synthesis of an Acetylated Cyclosporine Analogue Intermediate Using a Phosphonium Salt Compound Through a Wittig Reaction

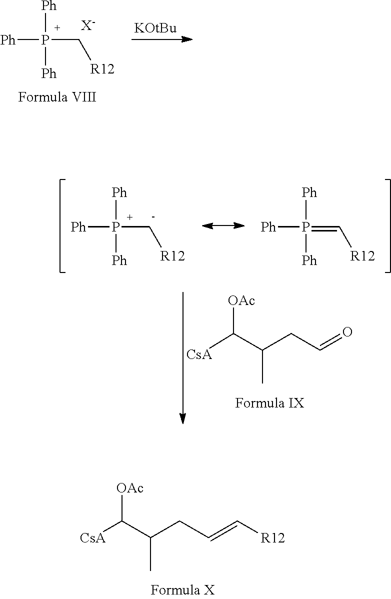

Where X is a halide (including but not limited to Cl, Br, and I), and R12 is a saturated or unsaturated. straight or branched aliphatic chain, optionally containing a substituent selected from the group of ketones, hydroxyls, nitriles, carboxylic acids, esters and 1,3-dioxolanes; an aromatic group, optionally containing a substituent selected from the group of halides, esters and nitro; or a combination of the aforementioned saturated or unsaturated, straight or branched aliphatic chain and the aformentioned aromatic groups.

### Example 3

### Synthesis of Compound 404-20 Using a Phosphonium Salt Compound Through a Wittig Reaction

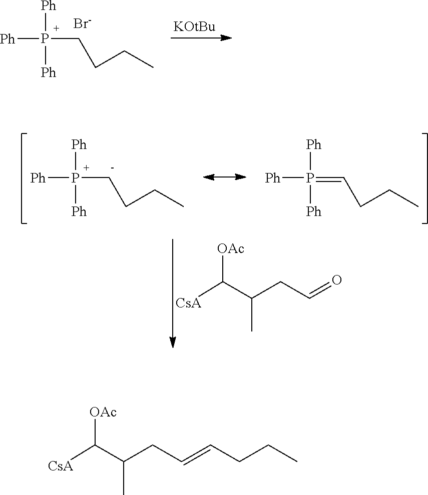

As an illustrative example, an oven dried 250 mL flask is charged under argon atmosphere with triphenylbutylphosphonium bromide (6.0 mmol) and 40 mL anhydrous tetrahydrofuran (THF). The suspension is cooled to 0° C. and potassium tert-butoxide (6.0 mmol) is added to obtain an orange color. The reaction is stirred at ambient temperature for 1-2 hours, followed by addition of CsA-aldehyde (2.0 mmol, dissolved in 20 mL anhydrous THF). Stirring is continued for 3 hours at room temperature. The reaction is quenched with 10 mL sat. NH₄Cl and 20 mL ice-water. The layers are separated and the aqueous phase is extracted with EtOAc. The organic layers are combined, washed with brine and dried over Na₂SO₄. The solvent is removed and the crude product is purified over silica gel (hexane/acetone 3:1).

Using Reaction 3, the following compounds are further examples of the compounds that may be synthesized.

| | | MS | |
|---|---|---|---|
| Compound | Starting Material | (Na⁺) | Remarks |
| 404-16 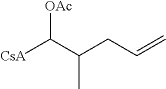 | 404-09 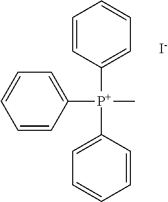 [SMILES: C[P+](c1ccccc1)(c1ccccc1)c1ccccc1 ; [I-]] | 1252.9 | |
| 404-19 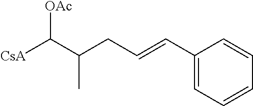 | 404-08 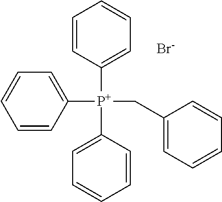 [SMILES: c1ccc(C[P+](c2ccccc2)(c2ccccc2)c2ccccc2)cc1 ; [Br-]] | 1328.9 | |
| 404-20 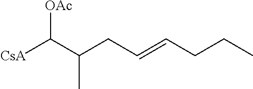 | 404-14 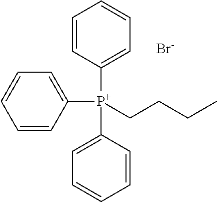 [SMILES: CCCC[P+](c1ccccc1)(c1ccccc1)c1ccccc1 ; [Br-]] | 1294.9 | |
| 404-30 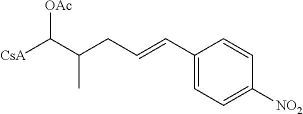 | 404-12 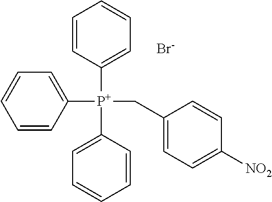 [SMILES: O=[N+]([O-])c1ccc(C[P+](c2ccccc2)(c2ccccc2)c2ccccc2)cc1 ; [Br-]] | 1373.9 | stirred at 60° C. over night |
| 404-31 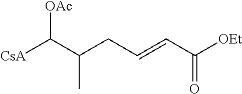 | 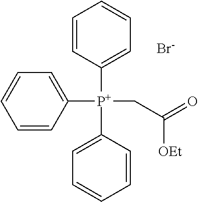 [SMILES: CCOC(=O)C[P+](c1ccccc1)(c1ccccc1)c1ccccc1 ; [Br-]] | 1324.9 | stirred at 60° C. for 2 days |
| 404-33 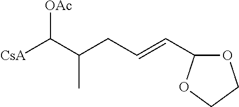 | 404-29 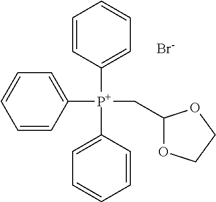 [SMILES: c1ccc([P+](CC2OCCO2)(c2ccccc2)c2ccccc2)cc1 ; [Br-]] | 1325.0 | |
| 404-40 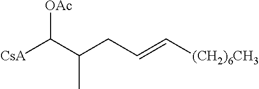 | 404-34 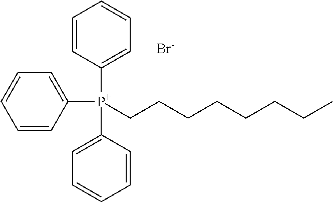 [SMILES: CCCCCCCC[P+](c1ccccc1)(c1ccccc1)c1ccccc1 ; [Br-]] | 1351.2 | |
| 404-43 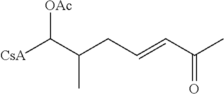 | 404-15 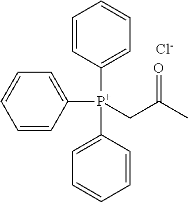 [SMILES: CC(=O)C[P+](c1ccccc1)(c1ccccc1)c1ccccc1 ; [Cl-]] | 1295.1 | stirred at reflux for 10 days |
| 404-59 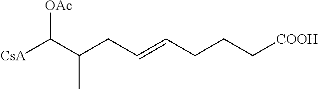 | 404-51 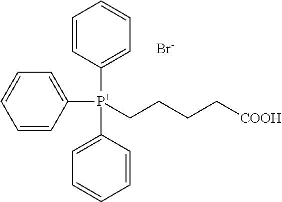 [SMILES: O=C(O)CCCC[P+](c1ccccc1)(c1ccccc1)c1ccccc1 ; [Br-]] | 1338.9 | 2 eq of KOtBu |
| 404-65 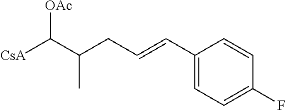 | 404-64 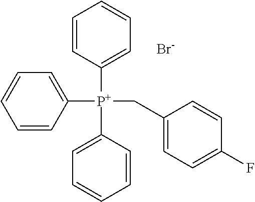 [SMILES: Fc1ccc(C[P+](c2ccccc2)(c2ccccc2)c2ccccc2)cc1 ; [Br-]] | 1347.1 | |
| 404-79 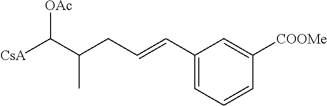 | 404-77 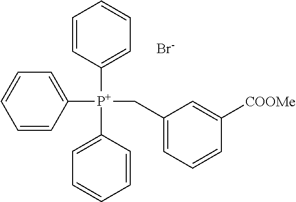 [SMILES: COC(=O)c1cccc(C[P+](c2ccccc2)(c2ccccc2)c2ccccc2)c1 ; [Br-]] | 1386.9 | stirred at RT over night |
| 404-89 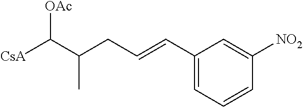 | 404-87 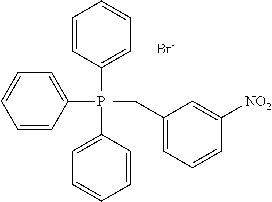 [SMILES: O=[N+]([O-])c1cccc(C[P+](c2ccccc2)(c2ccccc2)c2ccccc2)c1 ; [Br-]] | 1374.1 | stirred at RT for 2 days |
| 404-134 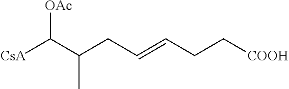 | 404-116 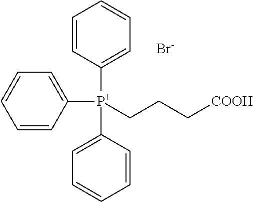 [SMILES: O=C(O)CCC[P+](c1ccccc1)(c1ccccc1)c1ccccc1 ; [Br-]] | 1325.0 | 2 eq of KOtBu; stirred at RT for 2 days |
| 404-163 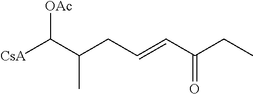 | 404-161 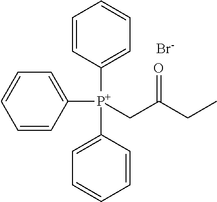 [SMILES: CCC(=O)C[P+](c1ccccc1)(c1ccccc1)c1ccccc1 ; [Br-]] | 1308.8 | stirred at reflux for 15 days |
| 404-187 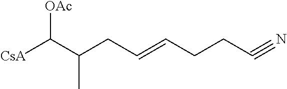 | 404-170 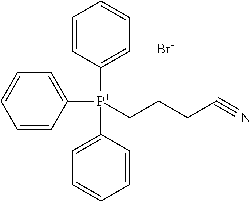 [SMILES: N#CCCC[P+](c1ccccc1)(c1ccccc1)c1ccccc1 ; [Br-]] | 1305.9 | stirred at RT over night |
| 416-04 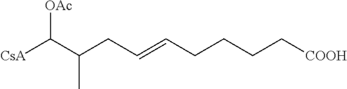 | 416-02 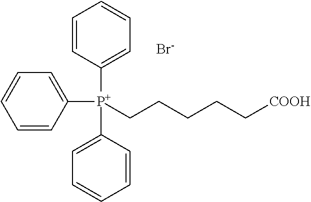 [SMILES: O=C(O)CCCCC[P+](c1ccccc1)(c1ccccc1)c1ccccc1 ; [Br-]] | 1353.0 | 2 eq of KOtBu |
| 416-09 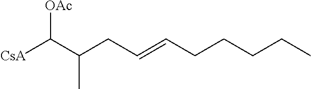 | 416-01 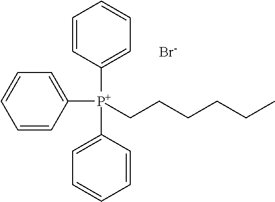 [SMILES: CCCCCC[P+](c1ccccc1)(c1ccccc1)c1ccccc1 ; [Br-]] | 1323.1 | |
| 420-40 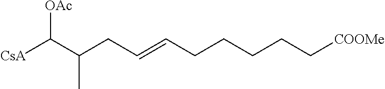 | 420-32 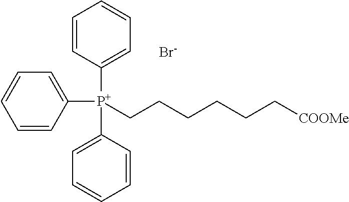 [SMILES: COC(=O)CCCCCC[P+](c1ccccc1)(c1ccccc1)c1ccccc1 ; [Br-]] | 1381.0 | stirred at RT over night |
| 420-85 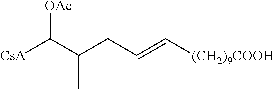 | 420-78 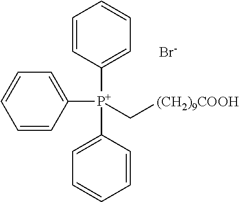 | 1423.1 | 2 eq of KOtBu |
| 420-89 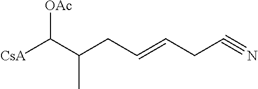 | 420-80 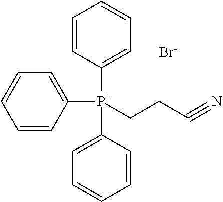 [SMILES: N#CCC[P+](c1ccccc1)(c1ccccc1)c1ccccc1 ; [Br-]] | 1291.9 | |
| 420-92 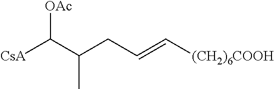 | 420-82 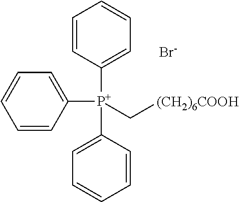 | 1381.1 | 2 eq of KOtBu |
| 420-96 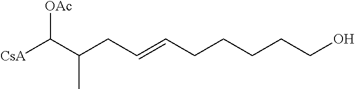 | 404-78 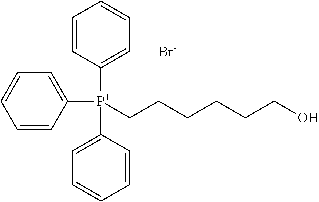 [SMILES: OCCCCCC[P+](c1ccccc1)(c1ccccc1)c1ccccc1 ; [Br-]] | 1338.9 | |
| 420-101 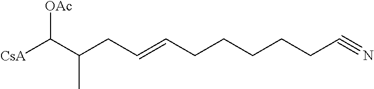 | 419-132 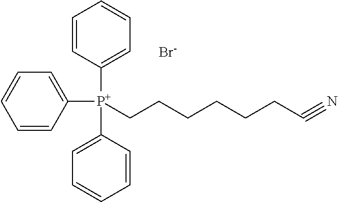 [SMILES: N#CCCCCCC[P+](c1ccccc1)(c1ccccc1)c1ccccc1 ; [Br-]] | 1347.9 | |

### Deacetylation

### Reaction 4

### Deacetylation of Acetylated Cyclosporine Analogues

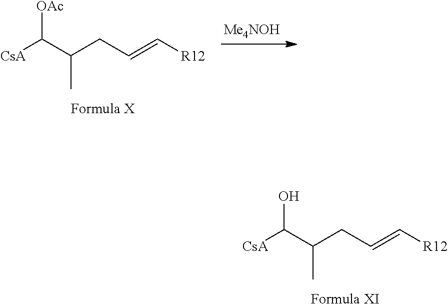

Where R12 is a saturated or unsaturated. straight or branched aliphatic chain, optionally containing a substituent selected from the group of ketones, hydroxyls, nitriles, carboxylic acids, esters, amides, acyl-protected amines and 1,3-dioxolanes; an aromatic group, optionally containing a substituent selected from the group of halides, esters, amines and nitro; or a combination of the aforementioned saturated or unsaturated, straight or branched aliphatic chain and the aforementioned aromatic groups.

### Example 4

### Synthesis of Compound 404-90 Though Deacetylation

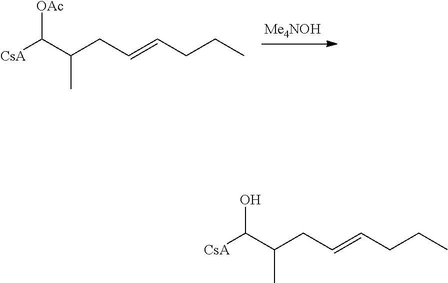

As an illustrative example, a solution of 404-20 (0.16 mmol) in 10 mL MeOH is combined with a solution of tetramethylammoniumhydroxide pentahydrate (0.47 mmol) in 2 mL H₂O. The mixture is stirred at room temperature for 2 days. The reaction is concentrated in vacuum and 5 mL H₂O are added. The reaction is extracted with EtOAc, the extract is washed with brine, dried over Na₂SO₄ and concentrated to dryness. The crude product is purified by reversed phase preparative HPLC.

Purification of deacetylated compounds is generally carried out over silica gel (hexane/acetone 2:1) or by Preparative HPLC. In the case of compounds 404-60, 404-137, 416-08, 420-98 and 420-100 (carboxylic acids), the reaction is acidified to pH 2-3 with 1 M HCl prior to extraction.

Using Reaction 4, the following compounds are further examples of the compounds that may be synthesized.

| | | MS |
|---|---|---|
| Compound | Starting Material | (Na⁺) |
| 404-22 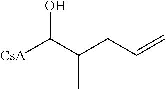 | 404-16 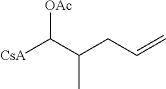 | 1210.9 |
| 404-25 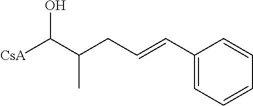 | 404-19 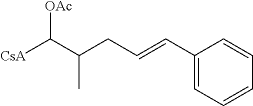 | 1287.0 |
| 404-36 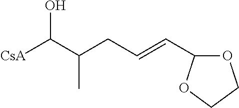 | 404-33 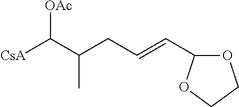 | 1283.0 |
| 404-44 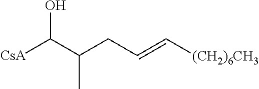 | 404-40 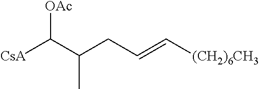 | 1309.1 |
| 404-58 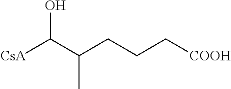 | 404-57 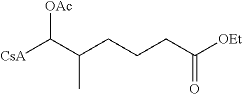 | 1257.1 |
| 404-60 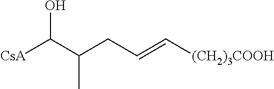 | 404-59 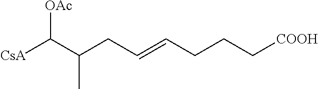 | 1297.1 |
| 404-61 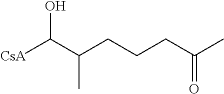 | 404-56 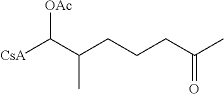 | 1255.1 |
| 404-66 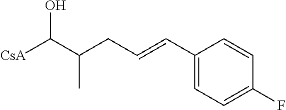 | 404-65 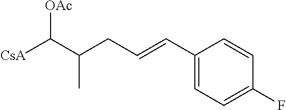 | 1305.1 |
| 404-81-1 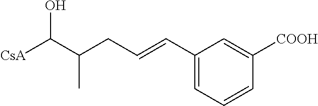 | 404-79-1 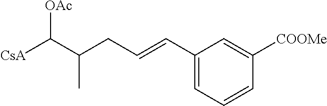 | 1331.1 |
| 404-81-2 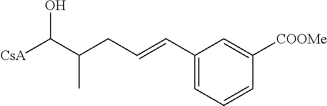 | 404-79-1 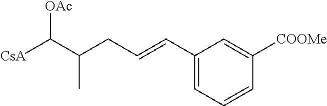 | 1345.1 |
| 404-85 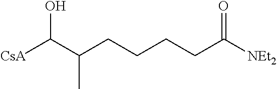 | 404-83 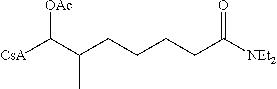 | 1326.2 |
| 404-90 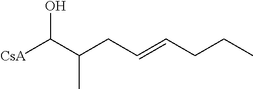 | 404-20 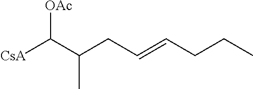 | 1253.0 |
| 404-96-1 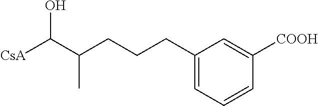 | 404-94 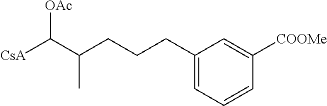 | 1333.0 |
| 404-96-2 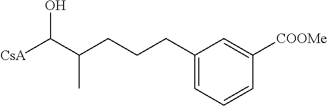 | 404-94 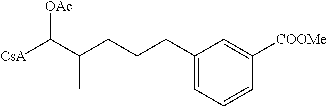 | 1347.0 |
| 404-97 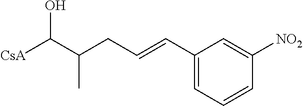 | 404-89 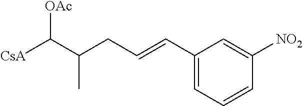 | 1331.9 |
| 404-125 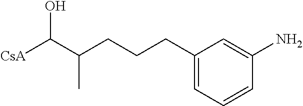 | 404-120 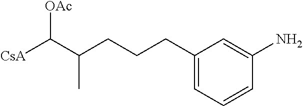 | 1304.0 |
| 404-130 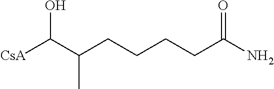 | 404-128 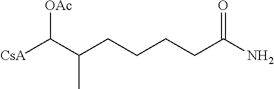 | 1270.1 |
| 404-132 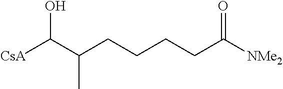 | 404-129 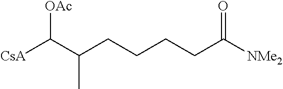 | 1298.0 |
| 404-137 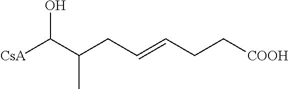 | 404-134 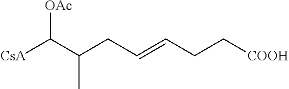 | 1283.0 |
| 404-154 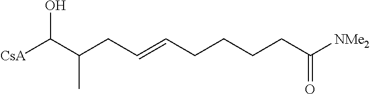 | 404-150 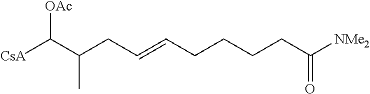 | 1338.1 |
| 404-157 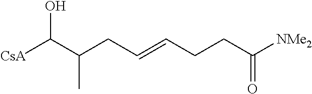 | 404-155 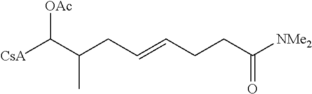 | 1310.0 |
| 404-173 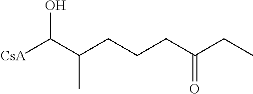 | 404-172 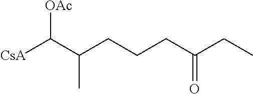 | 1268.9 |
| 404-194 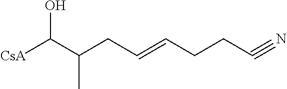 | 404-187 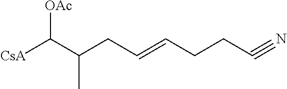 | 1263.9 |
| 416-08 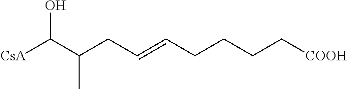 | 416-04 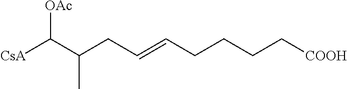 | 1311.0 |
| 416-13 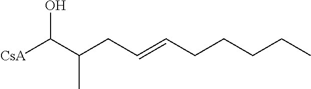 | 416-09 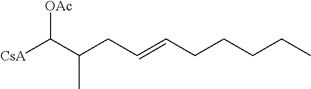 | 1281.1 |
| 420-17 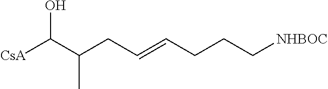 | 420-08-1 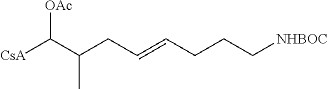 | 1368.0 |
| 420-30-1 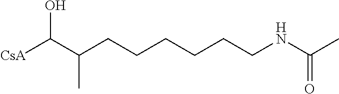 | 420-27 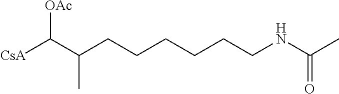 | 1312.0 |
| 420-43 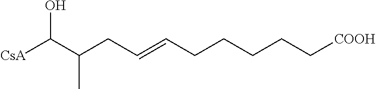 | 420-40 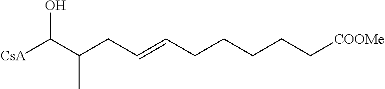 | 1324.9 |
| 420-47 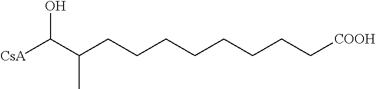 | 420-46 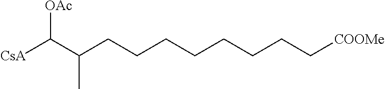 | 1327.0 |
| 420-98 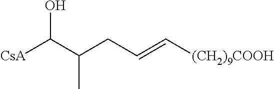 | 420-85 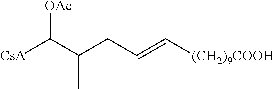 | 1381.1 |
| 420-100 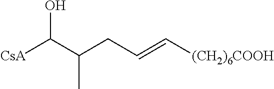 | 420-92 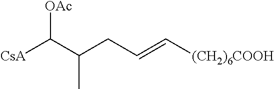 | 1339.1 |
| 420-102 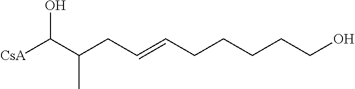 | 420-96 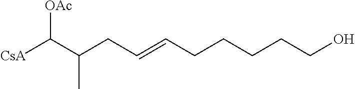 | 1297.0 |
| 420-108 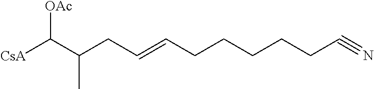 | 420-101 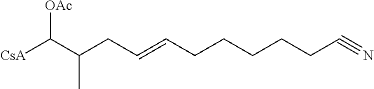 | 1305.9 |
| 420-117 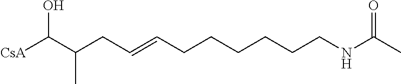 | 420-109-1 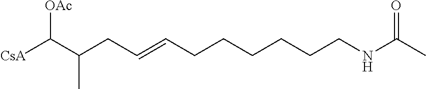 | 1352.1 |
| 420-120 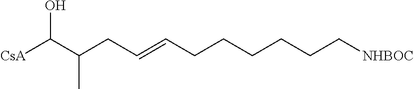 | 420-110-1 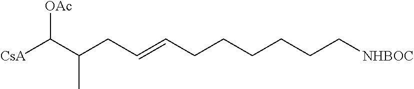 | 1410.0 |
| 420-122 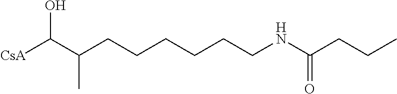 | 420-107-2 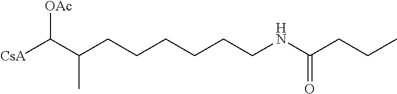 | 1340.0 |
| 420-124 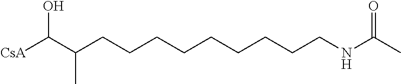 | 420-109-2 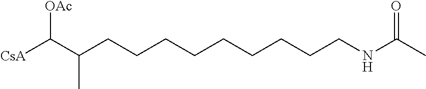 | 1354.0 |
| 420-125 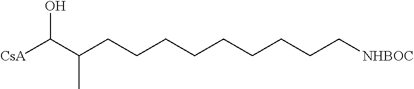 | 420-110-2 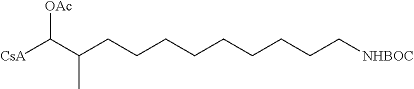 | 1412.0 |
| 420-126 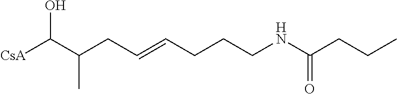 | 420-107-1 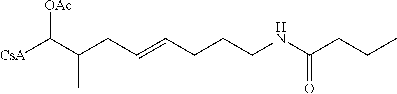 | 1337.9 |
| 420-131 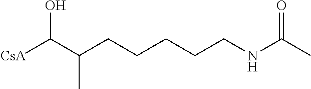 | 420-130 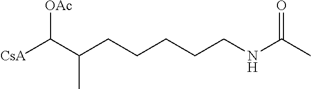 | 1297.9 |
| 420-132 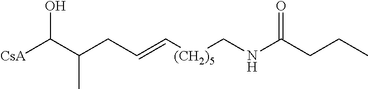 | 420-128-1 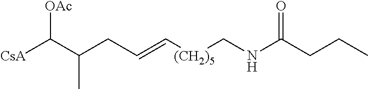 | 1380.0 |

### Hydrogenation of the Double Bond

The double bond can be hydrogenated under atmospheric pressure to obtain the saturated side chain. Functional groups such as hydroxyl, carbonyl and carboxyl are stable under these conditions and do not require protection. R′ represents either an acetyl group or hydrogen. In the case of α,β-unsaturated carbonyl compounds the double bond has to be reduced prior to deacetylation to avoid cyclization through a nucleophilic addition of the free hydroxy group on the activated double bond.

### Reaction 5

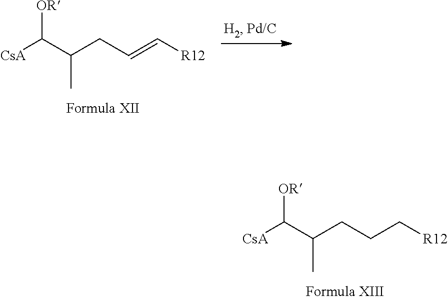

Where R12 is a saturated or unsaturated. straight or branched aliphatic chain, optionally containing a substituent selected from the group of ketones, hydroxyls, nitriles, carboxylic acids, esters, amides, acyl-protected amines and 1,3-dioxolanes; an aromatic group, optionally containing a substituent selected from the group of halides, esters, amines and nitro; or a combination of the aforementioned saturated or unsaturated, straight or branched aliphatic chain and the aforementioned aromatic groups, and R′ is either a H or an acetyl group.

### Example 5

### Synthesis of 404-56

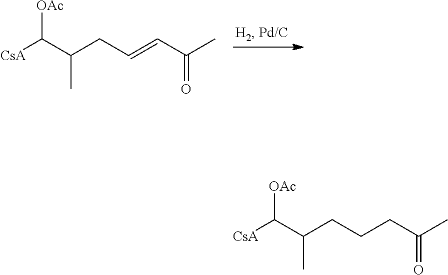

As an illustrative example, 404-43 (0.34 mmol) is dissolved in 40 mL anhydrous EtOH and 43 mg Pd/C (10%) and 0.2 mL acetic acid are added. The mixture is stirred under hydrogen at atmospheric pressure for 2 days. The reaction is filtered through Celite and is concentrated in vacuum. The crude product is purified by Preparative HPLC.

Using Reaction 5, the following compounds are further examples of the compounds that may be synthesized.

| Compound | Starting Material | MS (Na⁺) |
|---|---|---|
| 404-50 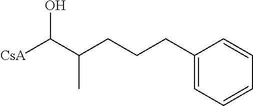 | 404-25 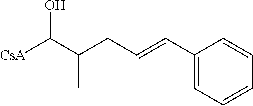 | 1289.1 |
| 404-56 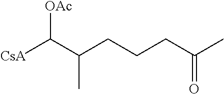 | 404-43 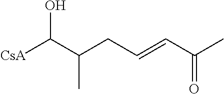 | 1297.0 |
| 404-57 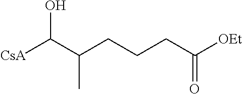 | 404-31 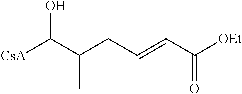 | 1327.1 |
| 404-63 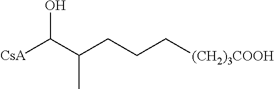 | 404-60 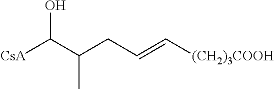 | 1299.1 |
| 404-74 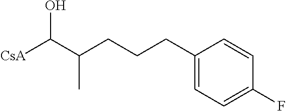 | 404-66 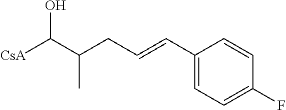 | 1307.1 |
| 404-92 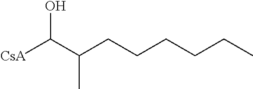 | 404-90 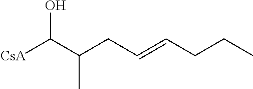 | 1255.1 |
| 404-94 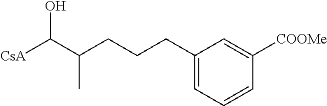 | 404-79 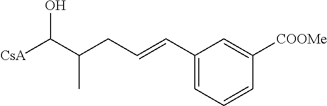 | 1388.9 |
| 404-168 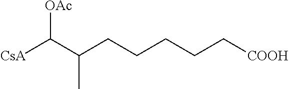 | 404-134 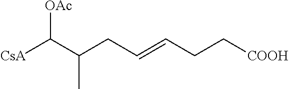 | 1326.8 |
| 404-172 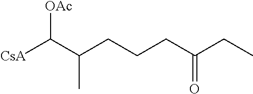 | 404-163 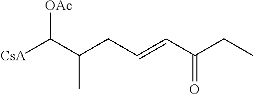 | 1310.9 |
| 420-19 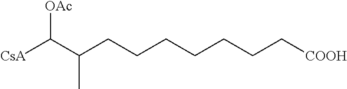 | 416-08 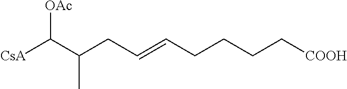 | 1313.0 |
| 420-46 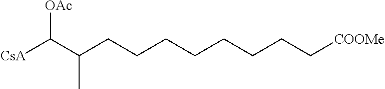 | 420-40 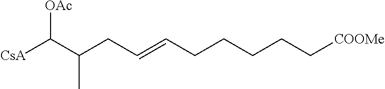 | 1383.1 |
| 420-68 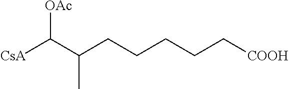 | 420-134 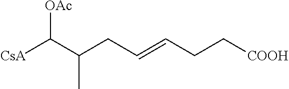 | 1326.9 |
| 420-106 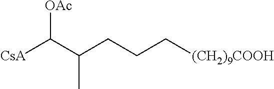 | 420-98 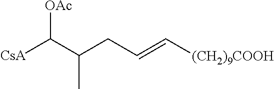 | 1383.1 |
| 420-111 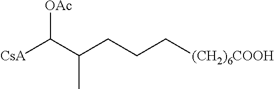 | 420-100 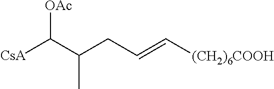 | 1341.0 |
| 420-112 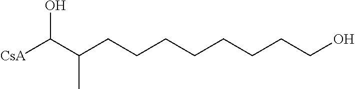 | 420-102 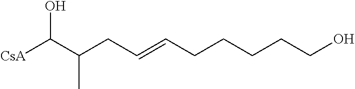 | 1298.9 |
| 420-130 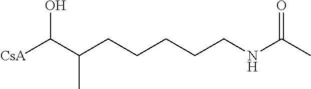 | 420-123 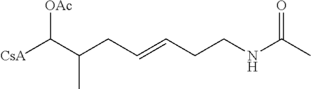 | 1340.0 |

### Reduction of the Nitrile Group

Reduction of the nitrile group to the corresponding primary amine can be achieved with nickel boride generated in situ from sodium borohydride (NaBH₄) and nickel(II)chloride (NiCl₂). Addition of a suitable trapping reagent leads to acyl-protected primary amines (carbamates or amides, respectively) and prevents the formation of secondary amines as an undesired side reaction. The double bond is partially reduced under the given conditions and a product mixture is obtained. Both, saturated and unsaturated protected amine compounds were isolated and purified. For reaction 420-123 the mixture was not separated. Instead, the mixture underwent catalytic hydrogenation to produce the fully saturated compound.

### Reaction 6

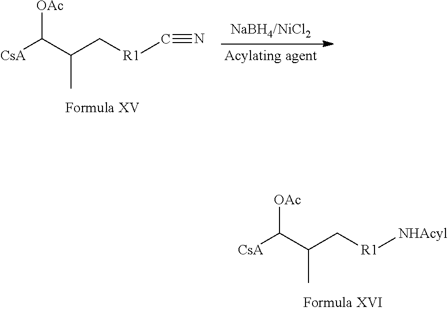

Where Acyl is any one of BOC, acetyl, or butyryl, acylating agent is any one of di-tert-butyldicarbonate, acetic anhydride, and butyric anhydride and R1 is a saturated or unsaturated straight chain or branched aliphatic group. It would be understood by one skilled in the art that the acylating agents described above may be replaced with a broad range of acylating agents to produce a similarly broad range of acyl-protected amines.

### Example 6

### Synthesis of 420-08

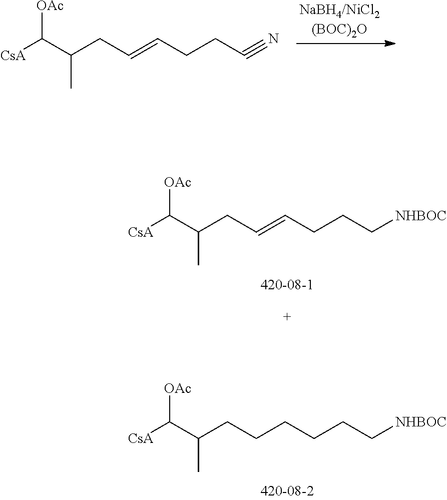

As an illustrative example, 404-187 (0.257 mmol) is dissolved in 15 mL methanol and cooled to 0° C. Di-tert-butyldicarbonate (0.514 mmol) and nickel(II)chloride (0.025 mmol) are added to give a clear solution. Sodiumborohydride (3.85 mmol) is added in portions over 1 hour. The resulting mixture is stirred at ambient temperature over night. Additional sodiumborohydride (1.95 mmol) is added at 0° C. and stirring is continued for 3 hours at room temperature. HPLC shows a mixture of 420-08-1 (carbamate compound) and 420-08-2 (carbamate compound with double bond reduced). The reaction is stirred for 30 minutes with diethylenetriamine (0.257 mmol) and is then concentrated in vacuum. The residue is taken up in 75 mL EtOAc, washed with 20 mL sat. NaHCO₃ solution and dried over Na₂SO₄. The solvent is removed in vacuum. The crude product is purified by Preparative HPLC. Using Reaction 6, the following compounds are further examples of the compounds that may be synthesized.

| | | Pro- | |
|---|---|---|---|
| | | tecting | MS |
| Compound | Starting Material | Reagent | (Na⁺) |
| 420-08-1 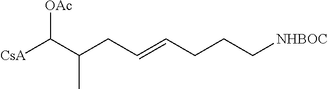 | 404-197 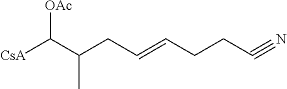 | di-tert- butyldi- carbon- ate | 1410.0 |
| 420-08-2 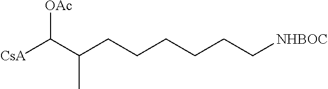 | 404-197 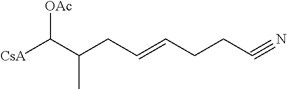 | di-tert- butyldi- carbon- ate | 1412.1 |
| 420-107-1 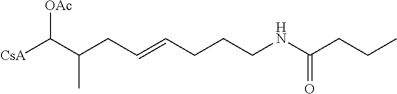 | 404-197 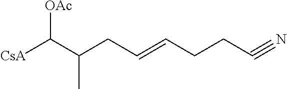 | butyric anhy- dride | 1379.9 |
| 420-107-2 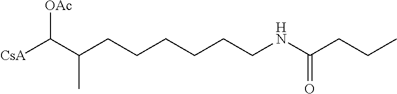 | 404-197 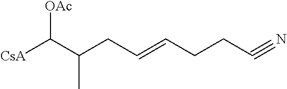 | butyric anhy- dride | 1382.1 |
| 420-109-1 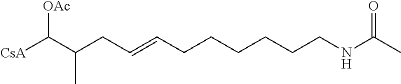 | 420-101 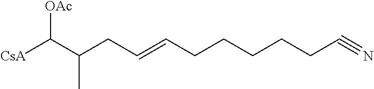 | acetic anhy- dride | 1394.1 |
| 420-109-2 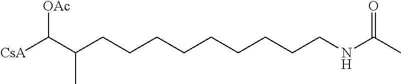 | 420-101 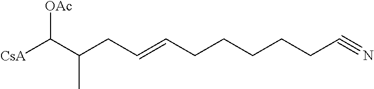 | acetic anhy- dride | 1396.1 |
| 420-110-1 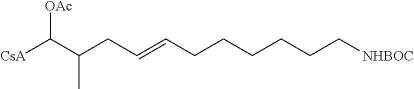 | 420-101 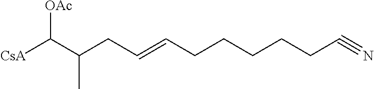 | di-tert- butyldi- carbon- ate | 1452.1 |
| 420-110-2 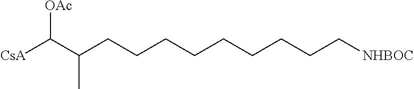 | 420-101 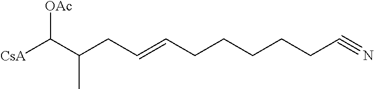 | di-tert- butyldi- carbon- ate | 1454.1 |
| 420-123 ¹ 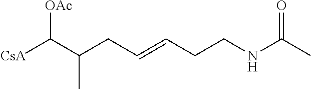 | 420-89 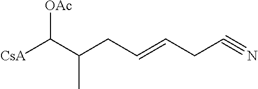 | acetic anhy- dride | 1337.9/ 1339.9 |
| 420-128-1 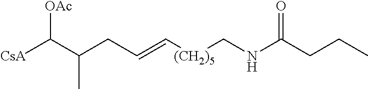 | 420-101 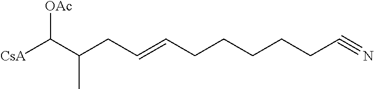 | butyric anhy- dride | 1422.1 |
| 420-128-2 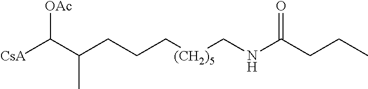 | 420-101 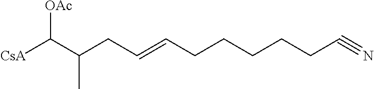 | butyric anhy- dride | 1424.1 |

¹ mixture not separated

### Amine Deprotection

The BOC protected amine (carbamate) can be converted into the free amine by acidic hydrolysis using trifluoroacetic acid (TFA).

### Reaction 7

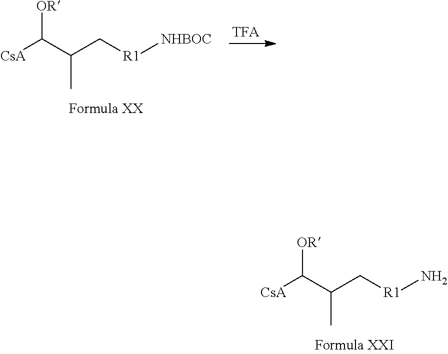

Where R1 is a saturated or unsaturated, straight or branched aliphatic chain, and R′ is either a H or an acetyl group.

### Example 7

### Synthesis of 420-23

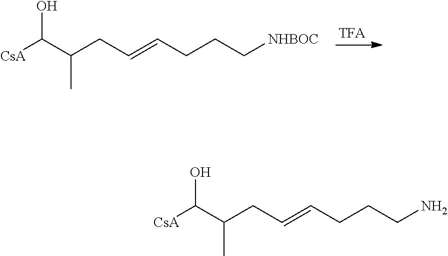

As an illustrative example, 420-17 (0.026 mmol) is dissolved in 4 mL anhydrous DCM and 2 mL trifluoroacetic acid is added at 0° C. The reaction is stirred at room temperature for 3 hours. Twenty 20 mL dichloromethane is added. The reaction mixture is washed with H₂O and sat. NaHCO₃ solution and is dried over Na₂SO₄. The solvent is removed and the crude product is purified by Preparative HPLC.

Using Reaction 7, the following compounds are further examples of the compounds that may be synthesized.

| Compound | Starting Material | MS (M + 1) |
|---|---|---|
| 420-23 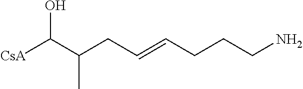 | 420-17 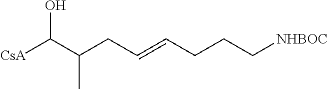 | 1246.0 |
| 420-25 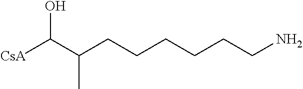 | 420-13 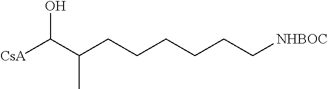 | 1290.0 |
| 420-129 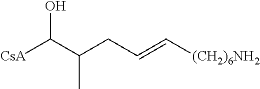 | 420-120 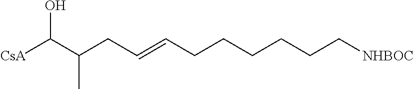 | 1288.0 |

### Protection of the Amino Group

The free amino function can be protected using a wide range of protecting groups using established methods. A broader range of protecting agents is available compared to the reductive introduction starting from the nitrile. Together, reactions 7 and 8 offer an alternate route to reaction 6 for the preparation of acyl-protected amino compounds.

### Reaction 8

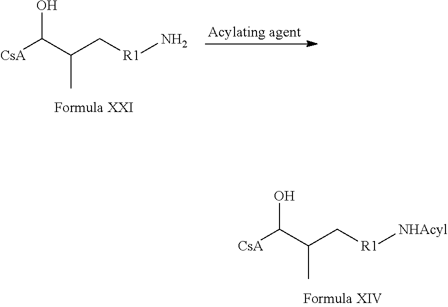

Where Acyl is any one of BOC, acetyl or butyryl, acylating agent is any one of di-tert-butyldicarbonate, acetic anhydride, butyric anhydride, It would be understood by one skilled in the art that a broad range of acylating agents including, dicarbonates, anhydrides and acyl halides can be employed to produce a broad range of acyl-protected amines, and R1 is a saturated or unsaturated straight chain or branched aliphatic group.

### Example 8

### Synthesis of 420-27

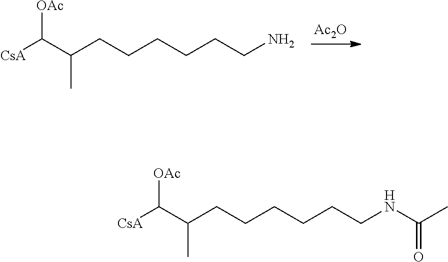

As an illustrative example, 420-25 (0.039 mmol) is dissolved in 3 mL anhydrous pyridine under nitrogen. The reaction is cooled to 0° C. and acetic anhydride (0.59 mmol) is added. The mixture is stirred at ambient temperature overnight. The solvent is removed in vacuum and the residue is taken up in 25 mL EtOAc. The reaction is washed with 2×10 mL 1 M HCl, 2×10 mL sat. NaHCO₃ solution and 10 mL brine and is dried over Na₂SO₄. The solvent is removed in vacuum to give the product as a colorless solid.

### Deprotection of Aldehyde

The 1,3-dioxolane moiety is converted into an aldehyde function through acidic hydrolysis.

### Reaction 9 and Example 9

### Synthesis of 404-47

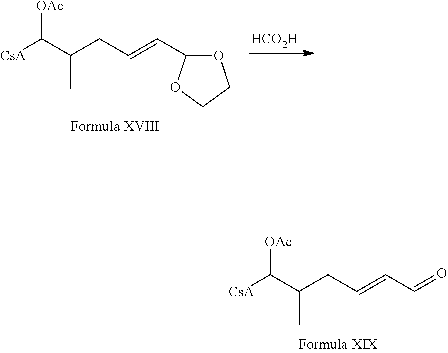

As an illustrative example, a solution of 404-33 (0.246 mmol) in 20 mL formic acid is stirred at room temperature for 45 minutes. One hundred mL ice-water and 200 mL sat. NaHCO₃ solution are added slowly to the reaction (strong foaming). The reaction is extracted with 2×150 mL EtOAc. The combined extracts are washed with sat. NaHCO₃ solution, water and brine and are dried over Na₂SO₄. The solvent is removed and the product is dried in vacuum.

### Reduction of the Nitro Group

The aromatic nitro compound is reduced to the aniline through catalytic hydrogenation. The reaction leads to the reduction of the double bond.

### Reaction 10 and Example 10

### Synthesis of 404-120

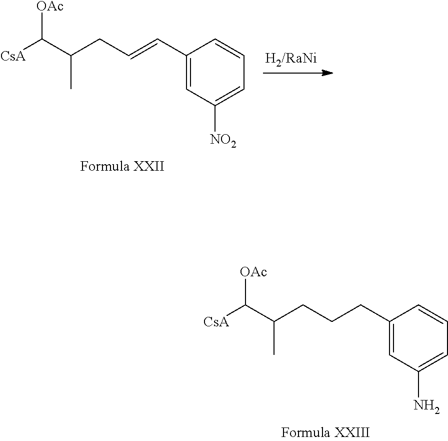

As an illustrative example, 404-89 (0.13 mmol) is dissolved in 2 mL ethanol and Raney-Nickel (0.18 g, 50% in H₂O, washed 3 times with ethanol, then suspended in 2 mL ethanol) and 0.1 mL acetic acid are added. The reaction is stirred at room temperature for 2 days. The reaction is filtered through Celite and the filter cake is washed with methanol. The filtrate is brought to dryness. The residue is taken up in EtOAc, washed with NaHCO₃ solution and brine and is dried over Na₂SO₄. The solvent is removed in vacuum. The crude product is purified over silica gel (hexane/acetone 2:1).

### Amide Synthesis

Amides are prepared from carboxylic acids by reaction of an amine with the corresponding acid chloride (Reaction 11). The synthesis can also proceed directly from the acid by use of appropriate coupling reagents, such as DCC and HOBt (Reaction 12).

### Reaction 11

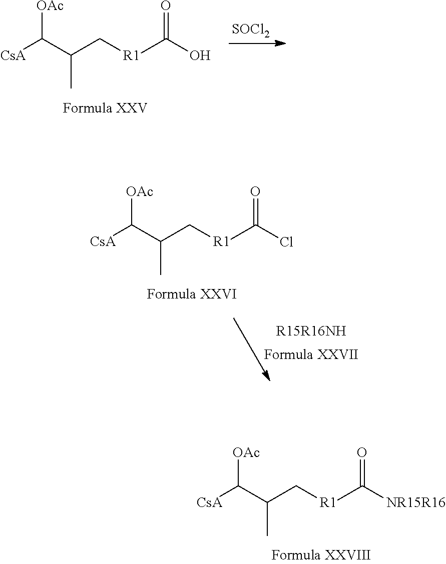

Where R1 is a saturated or unsaturated, straight or branched aliphatic chain, R15 and R16 are independently hydrogen or a saturated or unsaturated, straight or branched aliphatic chain, or where NR15R16 together forms a morpholinyl moiety.

### Example 11

### Synthesis of 404-85

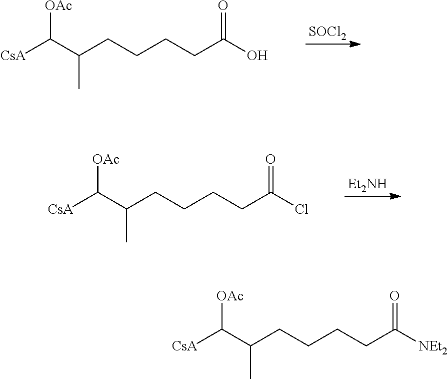

As an illustrative example, 365-73 (0.04 mmol) and thionylchloride (68 mmol) are combined under nitrogen atmosphere and are heated to reflux for 2 hours. The reaction is allowed to cool and is concentrated to dryness. Twenty mL toluene is added and the reaction is concentrated to dryness again (2 times). The residue is taken up in 5 mL anhydrous toluene and diethylamine (0.48 mmol) is added. The reaction is stirred at room temperature over night. Five mL H₂O are added and the mixture is extracted with 20 mL EtOAc. The extract is washed with brine and dried over Na₂SO₄. The solvent is removed in vacuum and the crude product is purified over silica gel (hexane/acetone 3:1).

Using Reaction 11, the following compounds are further examples of the compounds that may be synthesized.

| Compound | Starting Material | MS (Na⁺) | Amine |
|---|---|---|---|
| 404-83 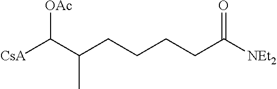 | 365-73 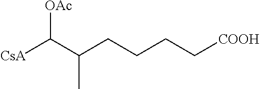 | 1368.2 | diethylamine |
| 404-128 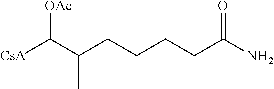 | 404-124 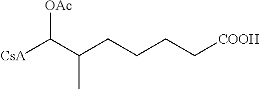 | 1311.9 | anhydrous ammonia ¹ |
| 404-129 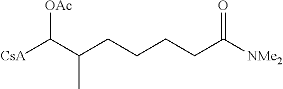 | 404-124 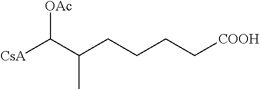 | 1340.1 | Dimethyl- amine ² |

¹ passed through reaction for 10 min at 0° C.;
² 2M solution in THF

### Reaction 12

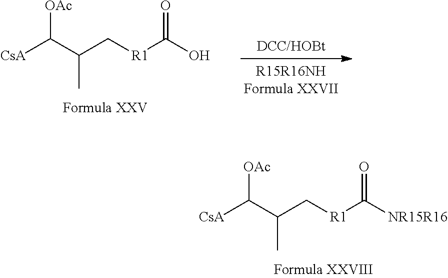

Where R1 is a saturated or unsaturated, straight or branched aliphatic chain, R15 and R16 are independently hydrogen or a saturated or unsaturated, straight or branched aliphatic chain, or where NR15R16 together forms a morpholinyl moiety.

### Example 12

### Synthesis of 420-104

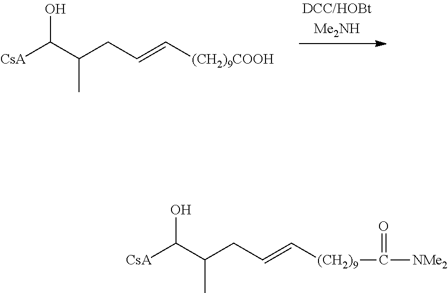

As an illustrative example, 420-98 (0.078 mmol) is dissolved in 10 mL anhydrous DCM under nitrogen atmosphere. Dicyclohexylcarbodiimide (DCC, 0.117 mmol) and 1-hydroxybenzotriazole hydrate (HOBt, 0.078 mmol) are added at 0° C. and the mixture is stirred for 15 minutes. Dimethylamine (0.78 mmol) is added to give a clear, colorless solution. The cooling bath is removed after 15 minutes and stirring is continued at ambient temperature for 5 days. The reaction is transferred to a separatory funnel and 20 mL DCM and 10 mL 0.5 M HCl are added. The organic layer is taken off, dried over Na₂SO₄ and concentrated to dryness. The residue is taken up in 10 mL acetonitrile. Undissolved solid is filtered off and the filtrate is concentrated in vacuum. The crude product is purified by Preparative HPLC.

Using Reaction 12, the following compounds are further examples of the compounds that may be synthesized.

| | | MS | |
|---|---|---|---|
| Compound | Starting Material | (Na⁺) | Amine |
| 404-150 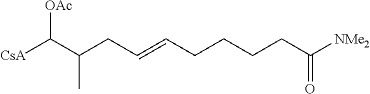 | 416-04 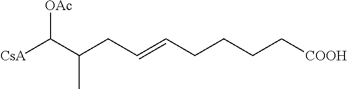 | 1380.1 | Dimethyl- amine ² |
| 404-155 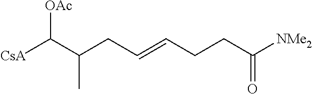 | 404-134 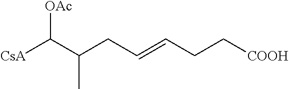 | 1352.1 | Dimethyl- amine ² |
| 404-156 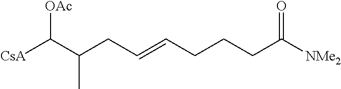 | 404-60 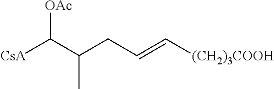 | 1324.1 | Dimethyl- amine ² |
| 404-162 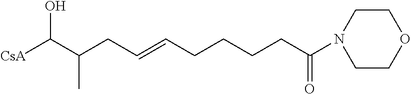 | 416-08 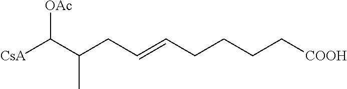 | 1379.9 | Morpholine |
| 404-164 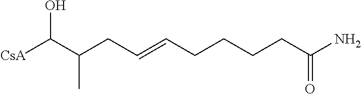 | 416-08 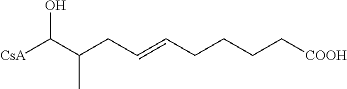 | 1309.8 | anhydrous ammonia ¹ |
| 404-178 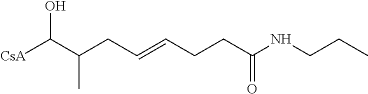 | 404-137 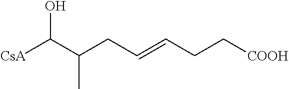 | 1323.9 | Propyl- amine |
| 420-104 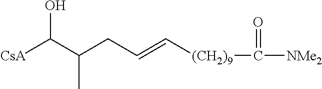 | 420-98 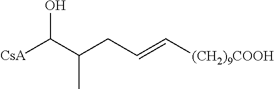 | 1408.1 | Dimethyl- amine ² |
| 420-114 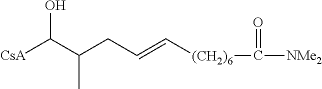 | 420-100 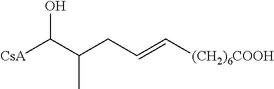 | 1366.0 | Dimethyl- amine ² |
| 420-121 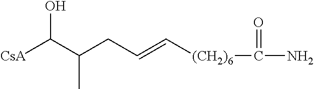 | 420-100 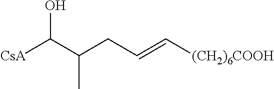 | 1338.0 | anhydrous ammonia ¹ |

¹ passed through reaction for 10 min at 0° C.;
² 2M solution in THF

### Esterification

Carboxylic acid esters are prepared from the corresponding carboxylic acids and an alcohol either using acidic catalysis (Reaction 13) or coupling reagents (DCC and DMAP, Reaction 14).

### Reaction 13

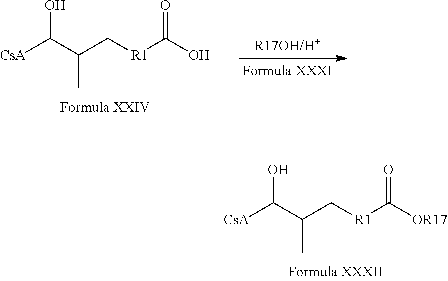

Where R1 is a saturated or unsaturated, straight or branched aliphatic chain, and R17 is a saturated or unsaturated, straight or branched aliphatic chain, optionally containing a halogen or hydroxyl substituent.

### Example 13

### Synthesis of 404-171

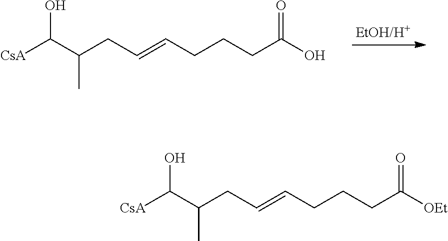

As an illustrative example, a mixture of 404-60 (0.059 mmol), 4 mL EtOH and 2 μL conc. H₂SO₄ is heated to reflux for 4 hours. The solvent is evaporated and the residue is taken up in acetonitrile. The crude product is purified by Preparative HPLC.

Using Reaction 13, the following compounds are further examples of the compounds that may be synthesized.

| Compound | Starting Material | MS (Na⁺) | Reagent |
|---|---|---|---|
| 404-171 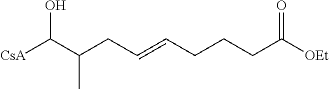 | 404-60 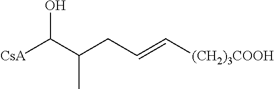 | 1368.2 | ethanol |
| 404-182 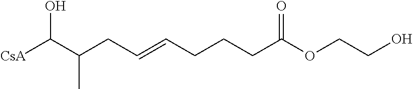 | 404-60 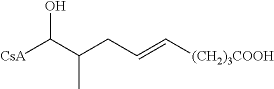 | 1311.9 | ethylene glycol ¹ |
| 420-103 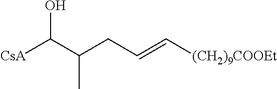 | 420-98 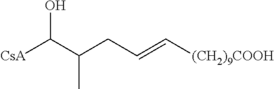 | 1409.1 | Ethanol |
| 420-113 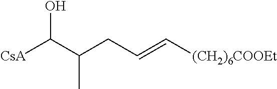 | 420-100 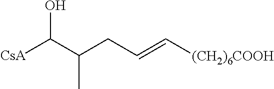 | 1366.9 | ethanol |

¹ 3 hours at 90° C.;
product extracted with EtOAc

### Reaction 14

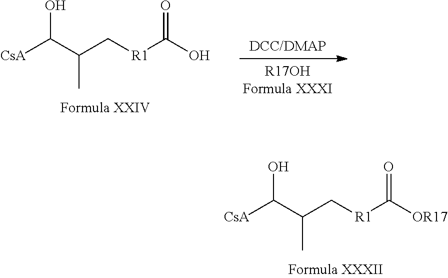

Where R1 is a saturated or unsaturated, straight or branched aliphatic chain, and R17 is a saturated or unsaturated, straight or branched aliphatic chain, optionally containing a halogen or hydroxyl substituent.

### Example 14

### 420-24

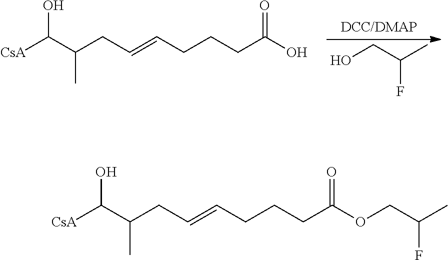

As an illustrative example, 404-60 (0.053 mmol) is dissolved in 4 mL anhydrous DCM and cooled to 0° C. under nitrogen atmosphere. Dimethylaminopyridine (DMAP, 0.005 mmol), 2-fluoropropanol (0.27 mmol) and dicyclohexylcarbodiimide (DCC, 0.058 mmol) are added and the reaction is stirred for 15 min at 0° C. The cooling bath is removed and stirring is continued over night at ambient temperature. 20 mL DCM are added, the reaction is then washed with H₂O and evaporated to dryness. The residue is taken up in 10 mL acetonitrile and filtered. The filtrate is concentrated in vacuum. The crude product is purified by Preparative HPLC.

### Alcohols

Besides direct synthesis in the Wittig reaction, alcohols are obtained through a number of reactions. Reduction of a carbonyl group with sodium borohydride leads to primary (starting from aldehyde) or secondary (starting from ketone) alcohols, respectively.

Oxidation of a double bond through the hydroboration method can lead to a mixture of isomers. The reaction proceeds predominantly in anti-Markovnikov orientation. In the case of a terminal olefin the primary alcohol is the main product. An olefin can be converted into a diol through oxidation with hydrogen peroxide. Reaction of a carbonyl compound with a Grignard reagent leads to secondary (starting from aldehyde) and tertiary (starting from ketone) alcohols. This method allows for an extension of the carbon chain.

### Reaction 15

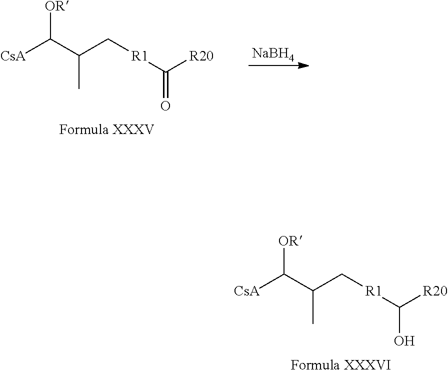

Where R′ is a H or acetyl, R1 is a saturated or unsaturated, straight or branched aliphatic chain, and R20 is a saturated or unsaturated, straight or branched aliphatic chain.

### Example 15

### Synthesis of 404-98

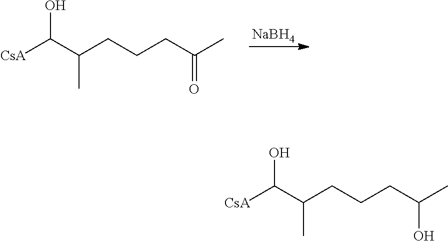

As an illustrative example, 404-61 (0.0365 mmol) is dissolved in 4.5 mL anhydrous EtOH under nitrogen atmosphere. Sodium borohydride (0.15 mmol, suspended in 0.5 mL anhydrous EtOH) is added at 0° C. and the resulting mixture is stirred at ambient temperature over night. Additional sodium borohydride (0.08 mmol) is added and stirring is continued over night. The reaction is quenched with 5 mL 1 M HCl under ice-bath cooling and is extracted with EtOAc. The extract is washed with brine, dried over Na₂SO₄ and concentrated to dryness. The crude product is purified by Preparative HPLC.

Using Reaction 15, the following compounds are further examples of the compounds that may be synthesized.

| Compound | Starting Material | MS (Na⁺) |
|---|---|---|
| 404-98 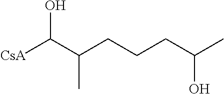 | 404-61 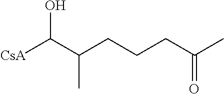 | 1256.9 |
| 404-195 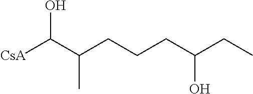 | 404-173 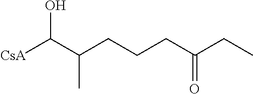 | 1271.0 |
| 404-198 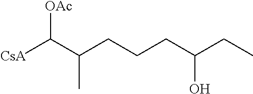 | 404-172 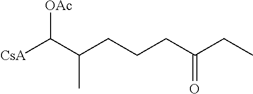 | 1313.0 |
| 420-09 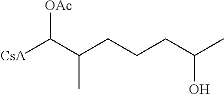 | 404-56 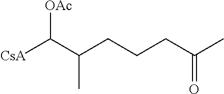 | 1298.9 |

### Reaction 16

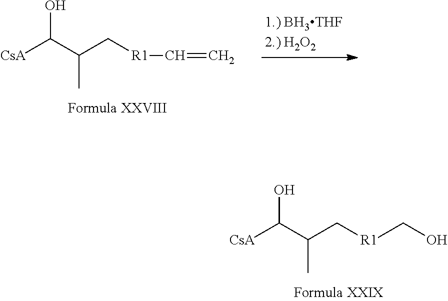

Where R1 is a saturated or unsaturated, straight or branched aliphatic chain.

### Example 16

### Synthesis of 420-28-1

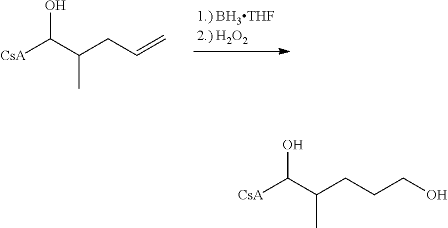

As an illustrative example, 404-16 (0.081 mmol) is dissolved under nitrogen atmosphere in 4 mL anhydrous THF. The reaction is cooled to 0° C. and BH₃·THF (1 M sol. In THF, 0.06 mmol) is added. The reaction is stirred at room temperature over night. HPLC shows the reaction is incomplete. Additional BH₃·THF (0.5 mmol) is added and stirring is continued for 4 hours at room temperature. The reaction is cooled to 0° C. and 1.0 mL 1 M NaOH and 0.30 mL 30% hydrogen peroxide solution are added. The mixture is stirred at room temperature over night. The reaction is extracted with 25 mL EtOAc. The extract is washed with brine, dried over Na₂SO₄ and concentrated to dryness. The product is purified by Preparative HPLC.

### Reaction 17

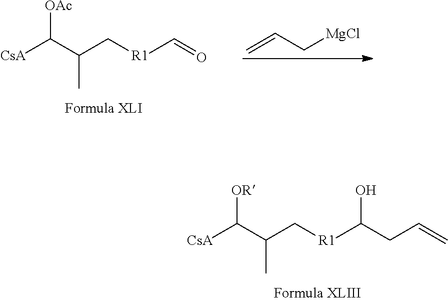

Where R1 is a saturated or unsaturated, straight or branched aliphatic chain, R′ is either a H or an acetyl group.

### Example 17

### Synthesis of 420-49

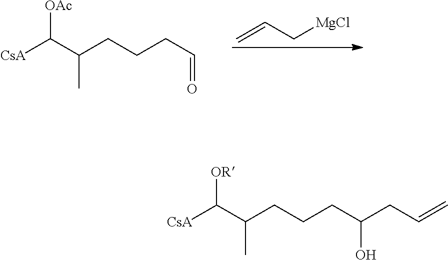

As an illustrative example, 420-49 (0.037 mmol) is dissolved under argon atmosphere in 5 mL anhydrous THF. The reaction is cooled to −70° C. and allylmagnesium chloride (1 M sol. In THF, 0.22 mmol) is added. The reaction is stirred for 15 minutes at −70° C. and is then allowed to come to room temperature. After 90 minutes the reaction is quenched with sat. NH₄Cl solution. The reaction is extracted with 25 mL EtOAc. The extract is washed with brine, dried over Na₂SO₄ and concentrated to dryness. The product is purified by Preparative HPLC. A mixture of acetylated and deacetylated compound is obtained.

### Reaction 18

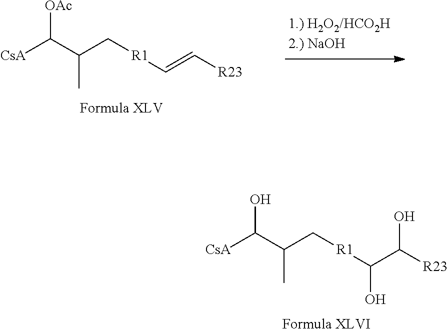

Where R1 is a saturated or unsaturated, straight or branched aliphatic chain, and R23 is a saturated or unsaturated, straight or branched aliphatic chain.

### Example 18

### Synthesis of 404-126

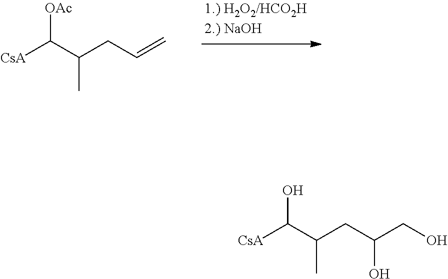

As an illustrative example, 404-16 (0.054 mmol) is dissolved in 1 mL formic acid and hydrogen peroxide (30% aqueous solution, 0.52 mmol) is added. The reaction is stirred at room temperature over night and is then concentrated to dryness. The residue is dissolved in 25 mL EtOAc, washed with sat. NaHCO₃ solution and dried over Na₂SO₄. The solvent is removed in vacuum. The reaction is taken up in 9 mL THF and 3 mL 1 M NaOH, and is stirred for 4 hours at room temperature. The solvent is removed and the residue is partitioned between 25 mL EtOAc and 5 mL H₂O. The organic layer is washed with brine and dried over Na₂SO₄. The solvent is evaporated and the crude product is purified by Preparative HPLC.

### Example 19

### Immunosuppression and Cyclophilin Isomerase Inhibition

### Immunosuppressive Potency

The immunosuppressive potency of test compounds was assessed by measuring their ability to inhibit the proliferation of human lymphocytes in cell culture. Lymphocytes were isolated from blood of normal human volunteers by Ficoll-gradient centrifugation and stained with 2 μg/ml carboxyfluoroscein diacetate succinimydyl ester (CFSE), a fluorescent cell division tracer molecule. Cells were stimulated through the CD3/T-cell receptor by seeding cells at 300,000/well into 96-well flat-bottom, high-binding plates coated with 1 μg/ml UCHT-1 anti-human CD3 antibody. Test compounds were prepared first as 10 mg/ml stock solutions in dimethylsulfoxide (DMSO). Test solutions were prepared by 500-fold dilution of the DMSO stock solutions, then 3-fold serial dilutions in cell culture medium (RPMI+5% FBS+penicillin-steptomycin) for a total of 7 concentrations per compound. Test solutions were added in equal volume to the culture wells containing cells to achieve final concentrations after dilution of 13.7 ng/ml-10,000 ng/ml. The reference compound, CsA, was prepared similarly but at concentrations ranging from 1.37-1,000 ng/ml. CsA was assayed in every experiment as a quality control for each experiment and as a reference comparison to the test compounds. Following 3 days incubation cells were stained with CD95-APC (lymphocyte activation marker) and analyzed by flow cytometry with a Becton Dickinson FACSCalibur. Percentage cell division was assessed in forward/side-scatter-gated lymphocytes by measuring the proportion of cells that underwent one or more cell divisions as determined by serial halving of CFSE intensity. The nondivided parent population was determined from samples maintained in culture without anti-CD3 stimulation. IC50 values for inhibition of cell division were determined by nonlinear regression analysis. Relative potency was calculated by normalizing IC50 values of test compounds to CsA.

Immunosuppressive potency was additionally analyzed by measuring the reduction in cell surface CD95 expression compared to vehicle controls.

### Cyclophilin D Inhibition Assay

A mitochondria swelling assay was used to measure the efficacy of NICAMs in blocking CyP-D and mitochondrial permeability transition. Under certain pathological conditions, mitochondria lose the ability to regulate calcium levels, and excessive calcium accumulation in the mitochondrial matrix results in the opening of large pores in the inner mitochondrial membrane. Nonselective conductance of ions and molecules up to 1.5 kilodaltons through the pore, a process called mitochondrial permeability transition, leads to swelling of mitochondria and other events which culminate in cell death. One of the components of the mitochondrial permeability transition pore (MPTP) is CyP-D. CyP-D is an immunophilin molecule whose isomerase activity regulates opening of the MPTP, and inhibition of the isomerase activity by CsA or CsA analogs inhibits creation of the MPTP. In general, mitochondria isolated from rat liver were exposed to calcium to induce MPTP opening in the absence or presence of test compounds, and calcium-induced swelling was measured as a reduction in light absorbance at 540 nm.

Mitochondria were isolated from fresh rat liver. Ice-cold or 4° C. conditions were used throughout all steps of the isolation. The liver was rinsed thoroughly and chopped in a small volume of isolation buffer (IB; 10 mM Hepes, 70 mM sucrose, 210 mM mannitol, 0.5 mM EDTA). Aliquots of the minced liver were homogenized in IB using a Teflon-glass Potter-Elvehjem tissue grinder and passed through a cell screen filter. The filtered homogenate was centrifuged at 600 g for 10 min, then the resulting supernatant centrifuged at 7000 g for 10 min. The supernatant was discarded, and the pellet resuspended in wash buffer (10 mM Hepes, 70 mM sucrose, 210 mM mannitol) and centrifuged a final time at 7000 g for 10 min. The supernatant was discarded, and the mitochondria-containing pellet suspended and stored on ice in 2 mL of respiration buffer (RB; 5 mM Hepes, 70 mM sucrose, 210 mM mannitol, 10 mM sodium succinate, 1 mM sodium phosphate dibasic).

Test compound solutions were prepared from 10 mg/ml stocks (dimethyl sulfoxide vehicle) first by diluting the test compound 1000× into respiration buffer #2 (RB2; 5 mM Hepes, 70 mM sucrose, 210 mM mannitol, 10 mM sodium succinate, 1 mM sodium phosphate dibasic, 1% fetal bovine serum, 2 μM rotenone), then by 3×-serial dilutions in RB2 to achieve test compound concentrations of 10000, 3333, 1111, 370, 123, 41, and 14 ng/mL. Polystyrene tubes and plates were used for all preparations.

Swelling assays were completed in a 96-well flat-bottom polystyrene plates. In each well a 10-μL aliquot of mitochondria suspension, equivalent to 100-200 μg total protein, was combined with 90 μL of test compound, incubated for 10 min, then the baseline absorbance measured on a plate reader (540 nm wavelength; A540). Swelling was induced by adding 5 μL of 4 mM calcium chloride to achieve a final calcium concentration of 190 μM. Mitochondria swelling was indicated by a decline in A540. A540 was measured immediately after calcium addition and at intervals up to 20 min, by which time no further reduction in A540 was observed. Duplicate samples were assayed for each test compound concentration.

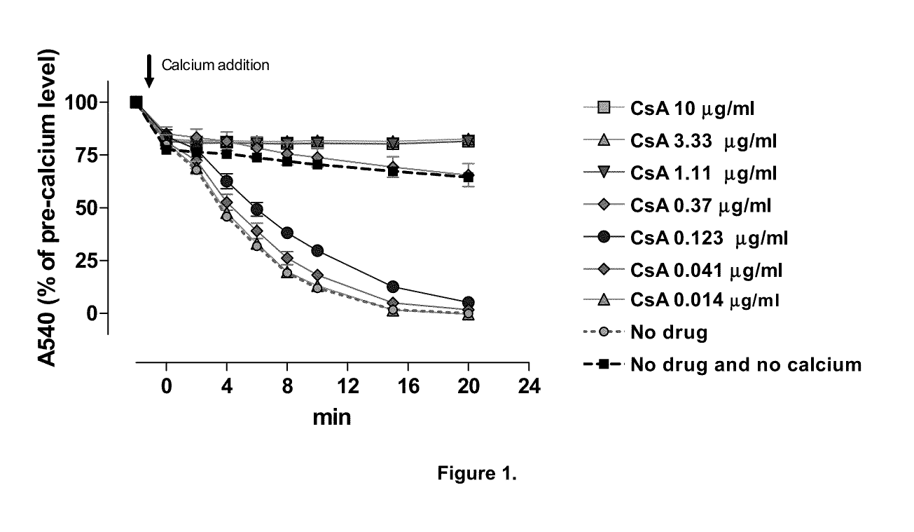

FIG. 1 shows the time course of mitochondrial absorbance following addition of calcium chloride in the absence or presence of CsA. CsA inhibited mitochondria swelling in a concentration-dependent manner, as indicated by blocking the calcium-induced decline in A540. Means and ranges of duplicate samples are shown.

TABLE 1
NICAM Compounds as Determined by Immunosuppressive Potency and
Cyp Binding Relative to CsA.

| | | Immuno- | CYP-D |
|---|---|---|---|
| | | suppression | Inhibition |
| | | (% Relative to | (% Potency Vs |
| Compound # | Compound structure | CsA) | CsA) |
| 404-26 | 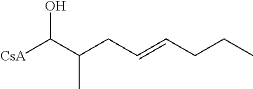 | 25 | 58.4 |
| 404-44 | 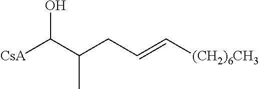 | 1 | 1.2 |
| 404-126 | 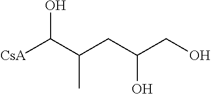 | <1 | 24.6 |
| 420-28 | 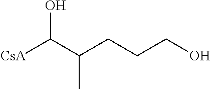 | <1 | 78.0 |
| 420-102 | 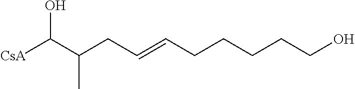 | 9 | 77.9 |
| 420-112 | 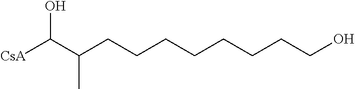 | 2 | 106.7 |
| 404-98 | 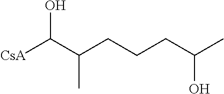 | 3 | 162.7 |
| 404-195 | 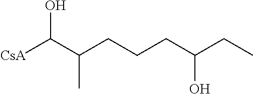 | 2 | 143.1 |
| 420-49-1 | 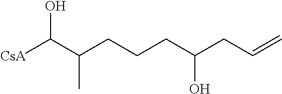 | 3 | 143.6 |
| 404-194 | 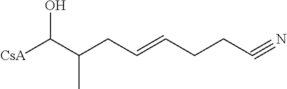 | 2 | 123.2 |
| 420-108 | 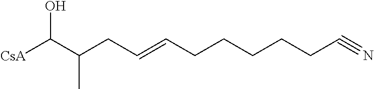 | 1 | 88.6 |
| 420-23 | 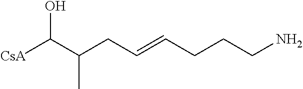 | <1 | 43.8 |
| 420-129 | 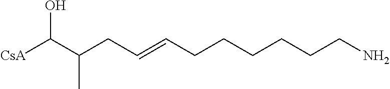 | <1 | 58 |
| 420-17 | 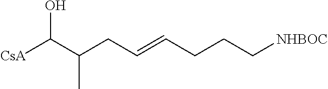 | 2 | 66.2 |
| 420-120 | 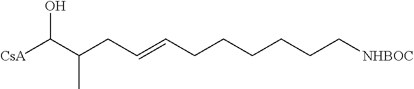 | 3 | 9.4 |
| 420-125 | 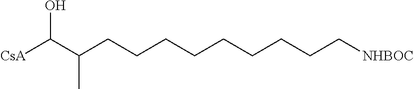 | 6 | 42.4 |
| 404-130 | 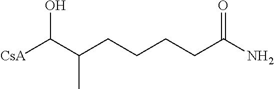 | <1 | 114.7 |
| 404-164 | 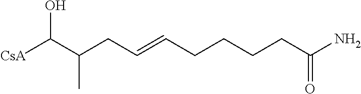 | 6 | 98.9 |
| 420-121 | 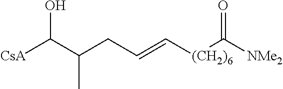 | 6 | 70.9 |
| 404-132 | 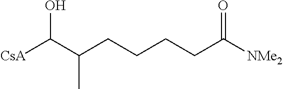 | <1 | 122.0 |
| 404-157 | 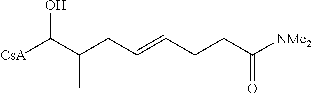 | <1 | 142.3 |
| 420-114 | 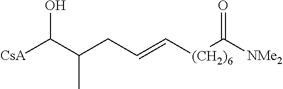 | <1 | 63.4 |
| 420-104 | 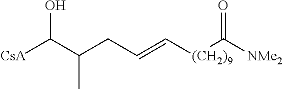 | 11 | 55.8 |
| 404-156 | 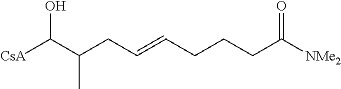 | 1 | 82.2 |
| 404-154 | 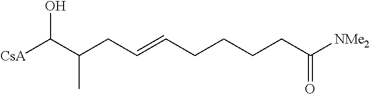 | 2 | 119.4 |
| 404-85 | 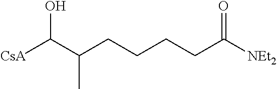 | 2 | 128.7 |
| 404-178 | 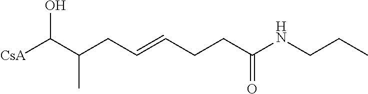 | <1 | 71.3 |
| 404-162 | 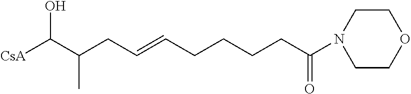 | <1 | 91.1 |
| 404-172 | 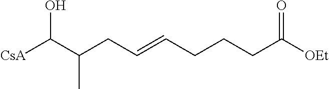 | 3 | 75.5 |
| 420-113 | 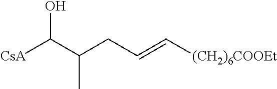 | <1 | 33.2 |
| 420-103 | 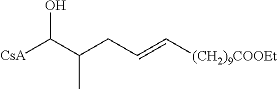 | 10 | 15.5 |
| 404-61 | 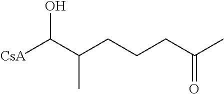 | 3 | 118.8 |
| 404-173 | 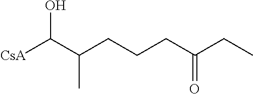 | 4 | 150.6 |
| 420-24 | 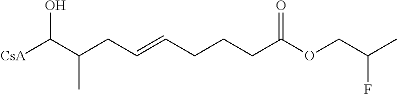 | <1 | 71.9 |
| 404-182 | 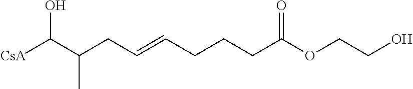 | 1 | 134.8 |
| 420-30-1 | 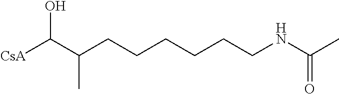 | 2 | 131.5 |
| 420-122 | 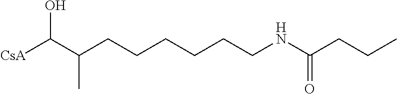 | 1 | 91.2 |
| 420-126 | 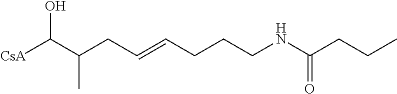 | 1 | 101.9 |
| 420-132 | 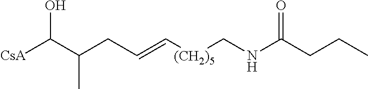 | 4 | 89.9 |
| 420-131 | 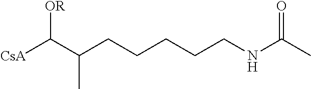 | <1 | 111.8 |
| 420-117 | 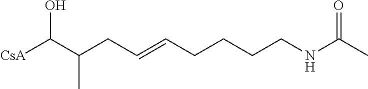 | 5 | 79.2 |
| 420-124 | 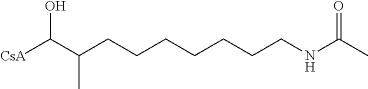 | 8 | 97.4 |
| 394-136 | 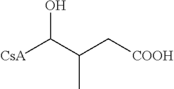 | <1 | 9.2 |
| 404-60 | 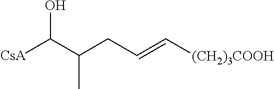 | <1 | 116.7 |
| 420-19 | 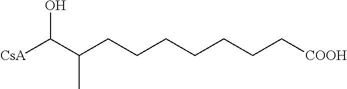 | <1 | 193.6 |
| 420-43 | 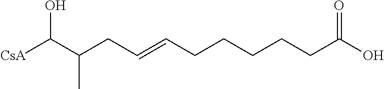 | 1 | 123.6 |
| 420-47 | 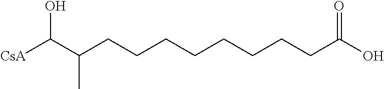 | 3 | 118.0 |
| 404-81-2 | 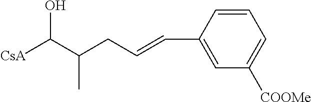 | 4 | 104.4 |
| 404-95 | 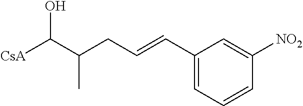 | 4 | 93.4 |
| 404-97 | 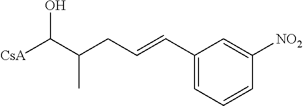 | 6 | 112.2 |
| 404-125 | 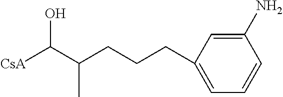 | 6 | 135.4 |
| 404-93-1 | 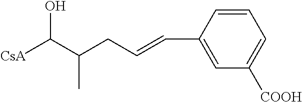 | <1 | 150.8 |
| 404-81-1 | 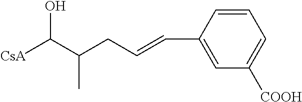 | 7 | 178.7 |

Table 1 sets out a number of identified NICAMS that are representative compounds that may be synthesized using Reactions 1-18 above. The NICAMS display<10% of the immunosuppressive potency of CsA while retaining >5% of the CyP binding of CsA. In many cases, the CyP binding of the NICAM has >50% of CsA, while reducing the immunosuppressive potency of the NICAM to <5% of that compared to CsA.

Although the present invention has been described by way of a detailed description in which various embodiments and aspects of the invention have been described, it will be seen by one skilled in the art that the full scope of this invention is not limited to the examples presented herein. The invention has a scope which is commensurate with the claims of this patent specification including any elements or aspects which would be seen to be equivalent to those set out in the accompanying claims.

1. A compound of Formula I: 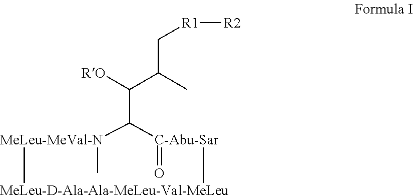
wherein
a. R′ is H or acetyl;
b. R1 is a saturated or unsaturated straight chain or branched aliphatic carbon chain from 2 to 15 carbon atoms in length; and
c. R2 is selected from the group consisting of:
i. a H;
ii. an unsubstituted, N-substituted, or N,N-disubstituted amide;
iii. a N-substituted or unsubstituted acyl protected amine;
iv. a carboxylic acid;
v. a N-substituted or unsubstituted amine;
vi. a nitrile;
vii. an ester;
viii. a ketone;
ix. a hydroxy, dihydroxy, trihydroxy, or polyhydroxy alkyl; and
x. a substituted or unsubstituted aryl.

2. A compound of Formula II: 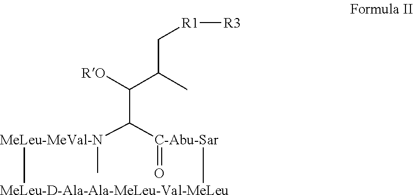
wherein
a. R′ is H or acetyl;
b. R1 is a saturated or unsaturated straight chain or branched aliphatic carbon chain from 2 to 15 carbon atoms in length; and
c. R3 is selected from the group consisting of:
i. a saturated or unsaturated. straight or branched aliphatic chain containing a substituent selected from the group consisting of hydrogen, ketones, hydroxyls, nitriles, carboxylic acids, esters and 1,3-dioxolanes;
ii. an aromatic group containing a substituent selected from the group consisting of halides, esters and nitro; and
iii. a combination of the saturated or unsaturated, straight or branched aliphatic chain of (i) and the aromatic group of (ii).

3. The compound of claim 1, wherein R2 is selected from the group consisting of 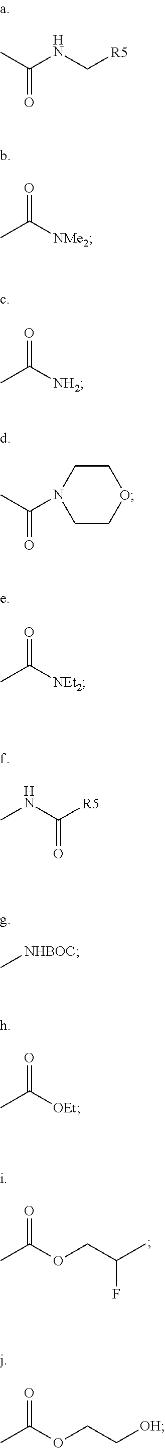 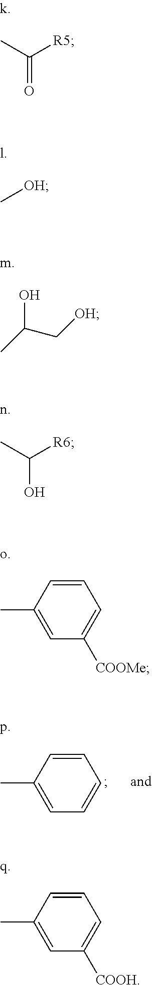
wherein
i. R5 is a saturated or unsaturated straight chain or branched aliphatic carbon chain between 1 and 10 carbons in length; and
ii. R6 is a monohydroxylated, dihydroxylated, trihydroxylated or polyhydroxylated saturated or unsaturated straight chain or branched aliphatic carbon chain between 1 and 10 carbons in length.

4. A compound of Formula IV: 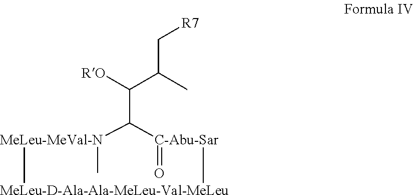
wherein
I. R′ is H or Acetyl; and
II. R7 is selected from the group consisting of:

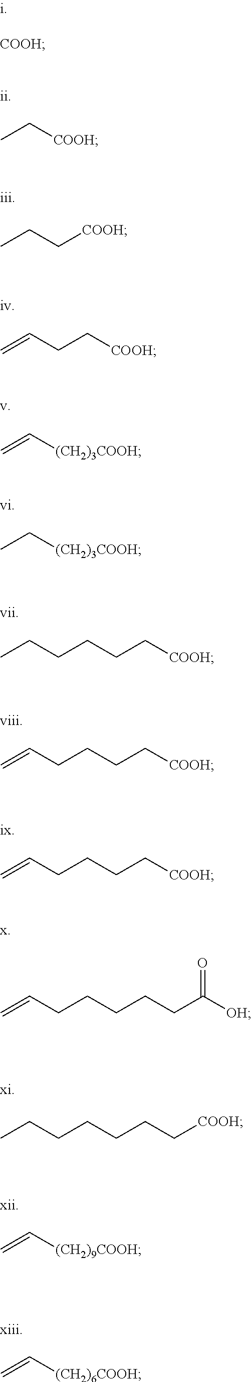

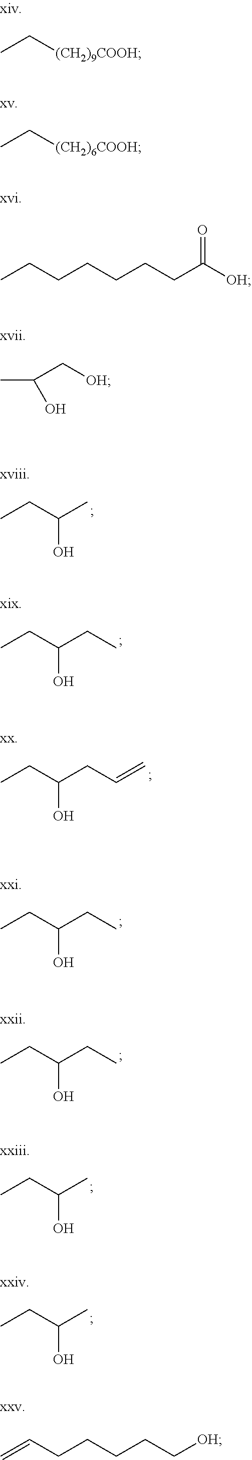

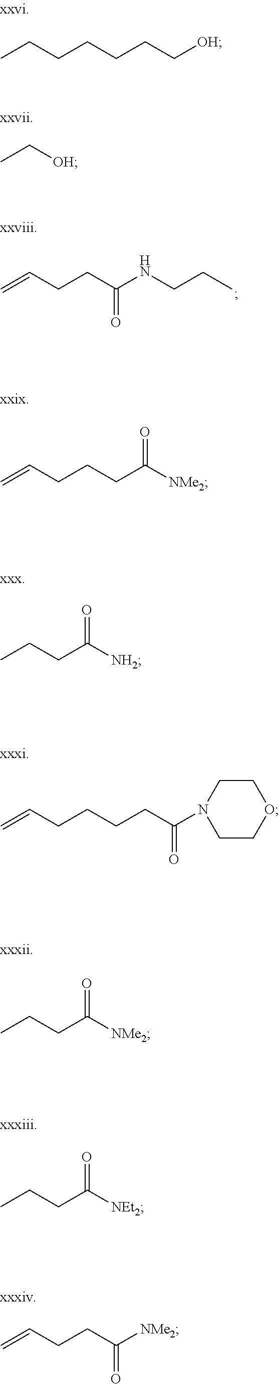

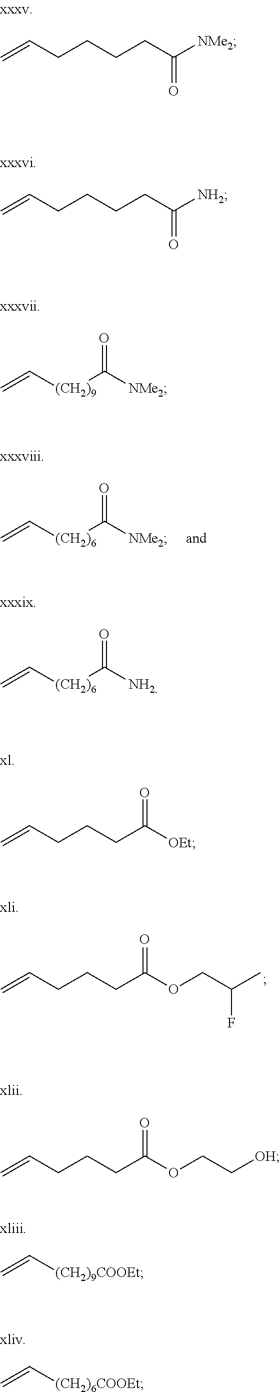

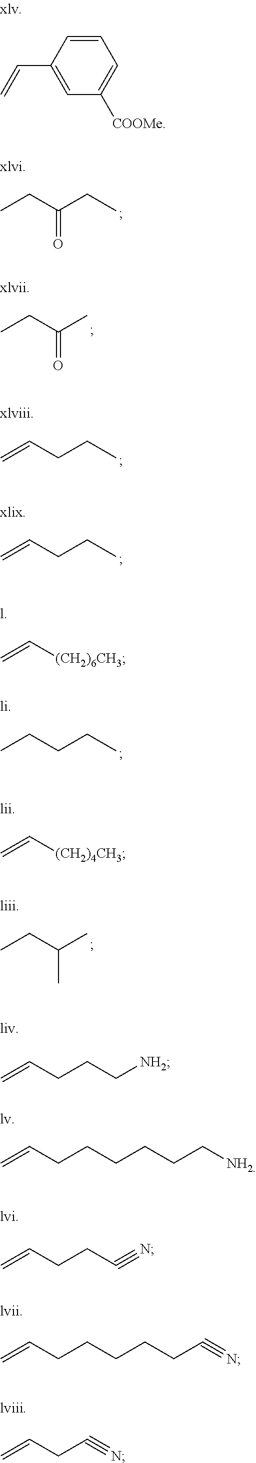

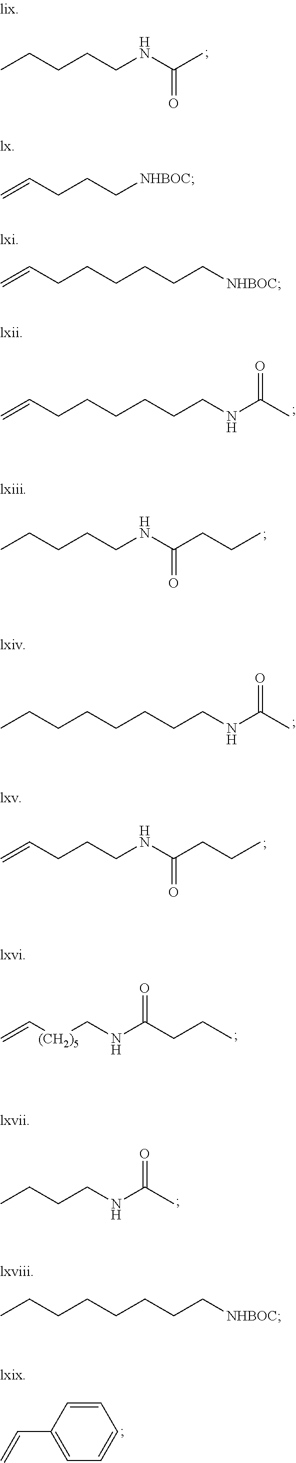

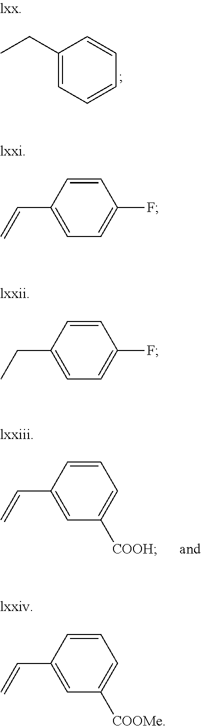

5. A process to produce a compound of Formula I: 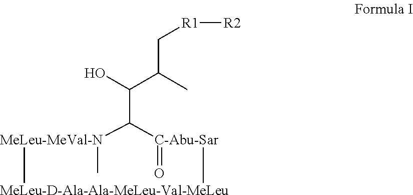
wherein R1 and R2 are as defined in claim 1,
comprising the steps of
a. reacting acetyl CsA aldehyde modified at amino acid 1 of Formula IX: 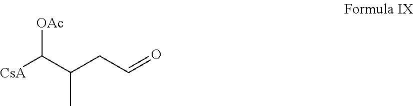
with a phosphonium salt of Formula VIII: 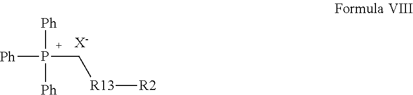
wherein R13 is a saturated or unsaturated straight chain or branched aliphatic carbon chain from 1 to 14 carbon atoms in length;
in the presence of a base to produce an acetylated compound of Formula X: 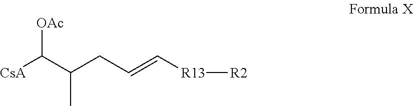
b. deacetylating the compound of Formula X using a base; and
c. where R1 is saturated, hydrogenating the double bond of the compound of Formula X by reacting the compound with a hydrogenating agent to produce a saturated analogue of Formula I.

6. The process of claim 5, wherein R2 is selected from the group consisting of 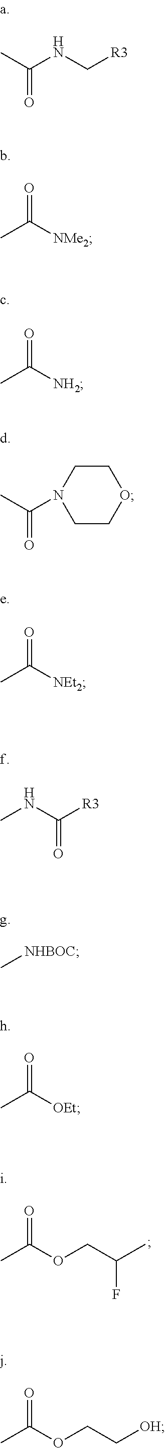 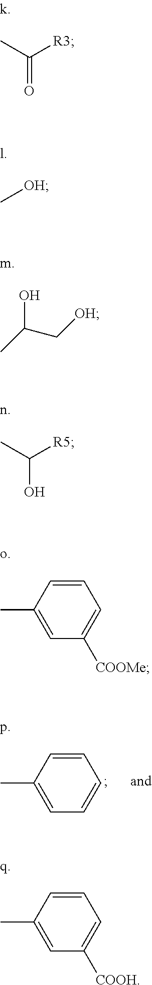
wherein
i. R5 is a saturated or unsaturated straight chain or branched aliphatic carbon chain between 1 and 10 carbons in length; and
ii. R6 is a monohydroxylated, dihydroxylated, trihydroxylated or polyhydroxylated saturated or unsaturated straight chain or branched aliphatic carbon chain between 1 and 10 carbons in length.

7. A process of producing a compound of the Formula XIV: 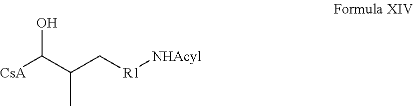
comprising the steps of
a. reacting the compound of Formula XV: 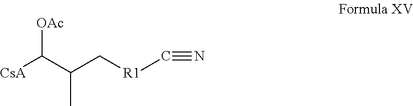
in the presence of a reducing agent and an acylating agent to produce acetylated compounds of Formula XVI: 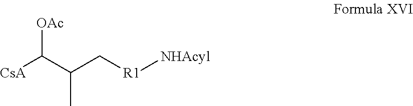
and
b. deacetylating the compound of Formula XVI using a base;
wherein R1 of Formulae XIV, XV and XVI is a saturated or unsaturated straight chain or branched aliphatic carbon chain between 2 and 15 carbons in length.

8. A process of producing a compound of the Formula XXI: 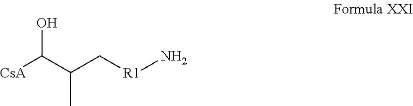
comprising the steps of
a. by dissolving the compound of Formula XX: 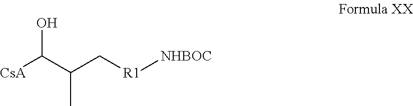
in an anhydrous solvent; and
b. reacting the solution with trifluoroacetic acid (TFA);
wherein R1 of Formulae XX and XXI is a saturated or unsaturated, straight chain or branched aliphatic carbon chain between 2 and 15 carbons in length.

9. A process of producing a compound of the Formula XIV: 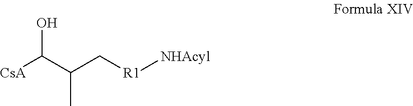
comprising the steps of
a. dissolving the compound of Formula XXI: 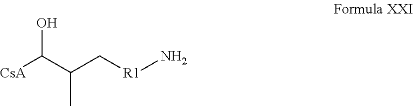
in anhydrous pyridine;
b. reacting the solution with acylating agent; and
c. removing the solvent to yield the compound of Formula XIV;
wherein R1 of Formulae XIV and XXI is a saturated or unsaturated straight chain or branched aliphatic carbon chain between 2 and 15 carbons in length.

10. A process of producing a compound of the Formula XXIV: 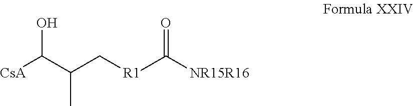
wherein
I. R1 is a saturated or unsaturated, straight or branched aliphatic carbon chain between 2 and 15 carbons in length; and
II. R15 and R16 are independently hydrogen or a saturated or unsaturated straight chain or branched aliphatic group; or where NR15R16 together forms a morpholinyl moiety;
comprising the steps of
a. by combining the compound of Formula XXV: 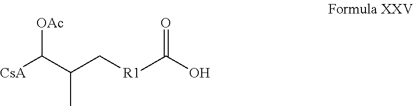
with thionylchloride to yield a residue of the Formula XXVI; 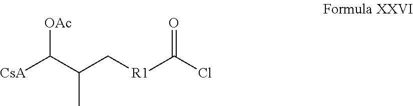
b. dissolving the residue in anhydrous solvent and reacting with a compound of the Formula XXVII:
R15R16NH Formula XXVII
to yield the compound of Formula XXVIII 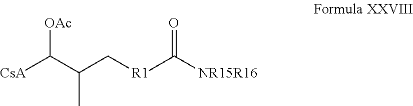
and
c. deacetylating the compound of Formula XXIV with a base.

11. A process of producing a compound of the Formula XXIV: 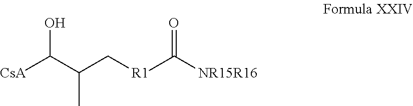
wherein
I. R1 is a saturated or unsaturated straight chain or branched aliphatic carbon chain between 2 and 15 carbons in length;
II. R15 and R16 are independently hydrogen or a saturated or unsaturated straight chain or branched aliphatic group; or where NR15R16 together forms a morpholinyl moiety;
comprising the steps of
a. dissolving the compound of Formula XXV: 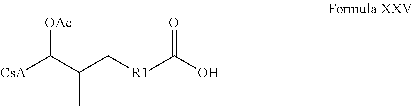
in anhydrous solvent under nitrogen;
b. reacting with dicyclohexylcarvodiimide, 1-hydroxybenzotriazole hydrate and the compound of the Formula XVIII;
R15R16NH Formula XXVII
and
c. deacetylating the compound of Formula XVIII with a base.

12. A process of producing a compound of the Formula XXXII: 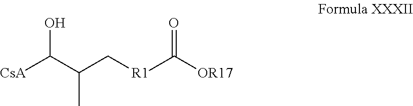
wherein
I. R1 is a saturated or unsaturated straight chain or branched aliphatic carbon chain between 2 and 15 carbons in length; and
II. R17 is a saturated or unsaturated straight chain or branched aliphatic group, optionally containing a halogen or hydroxyl substituent;
by reacting the compound of Formula XXX: 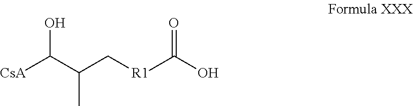
with a compound of Formula XXXI:
R17OH Formula XXXI
in the presence of an acid.

13. A process of producing a compound of the Formula XXVI: 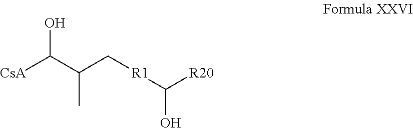
wherein
I. R1 is a saturated or unsaturated straight chain or branched aliphatic carbon chain between 2 and 15 carbons in length; and
II. R20 is a saturated or unsaturated straight chain or branched aliphatic group;
by reacting the compound of Formula XXXV: 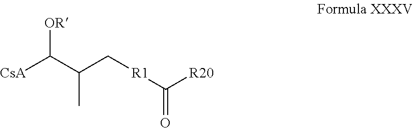
wherein R′ is optionally H or acetyl
with sodium borohydride; and
where R′ is acetyl, deacetylating the compound of Formula XXXV with a base.

14. A process of producing a compound of the Formula XXIX: 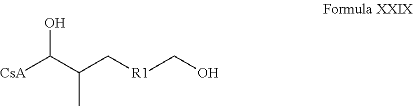
wherein R1 is a saturated or unsaturated straight chain or branched aliphatic carbon chain between 2 and 15 carbons in length;
by reacting the compound of Formula XXVIII: 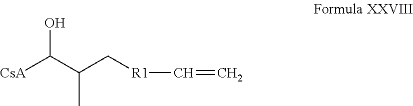
with borane-tetrahydrofuran and sodium peroxide.

15. A process of producing a compound of the Formula XLIII: 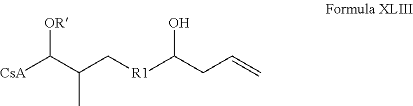
wherein
I. R′ is H or Acetyl; and
II. R1 is a saturated or unsaturated, straight or branched aliphatic chain between 2 and 15 carbons in length;
comprising the steps of:
reacting the compound of Formula XLI: 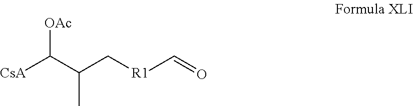
with the compound of Formula XLII; 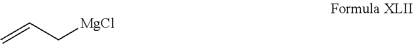
in an anhydrous solvent; and
deacetylating the compound of Formula XLII with a base.

16. A process of producing a compound of the Formula XLVI: 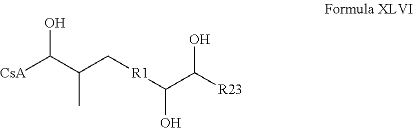
wherein
I. R1 is a saturated or unsaturated straight chain or branched aliphatic carbon chain from 2 to 15 carbon atoms in length; and
II. R23 is a saturated or unsaturated straight chain or branched aliphatic group;
comprising the steps of:
a. reacting the compound of Formula XLV 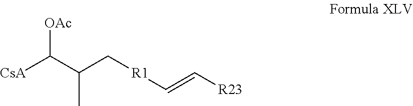
with hydrogen peroxide and formic acid;
b. reacting the product with a base to yield the compound of Formula XLVI; and
c. deacetylating the compound of Formula XLV with a base.

17. A pharmaceutical composition comprising a therapeutically effective amount of the compound of claim 1 and one or more pharmaceutical excipients.

18. A method of treating a cyclophilin mediated disease in a mammal comprising administering a therapeutically effective amount of the compound of claim 1 to the mammal under conditions to treat the cyclophilin mediated disease or injury.

19.-27. (canceled)

28. A pharmaceutical composition comprising a therapeutically effective amount of the compound of claim 2 and one or more pharmaceutical excipients.

29. A pharmaceutical composition comprising a therapeutically effective amount of the compound of claim 4 and one or more pharmaceutical excipients.

30. A method of treating a cyclophilin mediated disease in a mammal comprising administering a therapeutically effective amount of the compound of claim 2 to the mammal under conditions to treat the cyclophilin mediated disease or injury.

31. A method of treating a cyclophilin mediated disease in a mammal comprising administering a therapeutically effective amount of the compound of claim 4 to the mammal under conditions to treat the cyclophilin mediated disease or injury.

32. A method of treating a cyclophilin mediated disease or injury in a mammal comprising administering a therapeutic effective amount of the compound of claim 1 to the mammal under conditions to treat the cyclophilin mediated disease or injury, wherein the disease or injury is one or both of mediated by the over expression of cyclophilin and a congenital over expression of cyclophilin.

33. A method of treating a cyclophilin mediated disease or injury in a mammal comprising administering a therapeutic effective amount of the compound of claim 2 to the mammal under conditions to treat the cyclophilin mediated disease or injury, wherein the disease or injury is one or both of mediated by the over expression of cyclophilin and a congenital over expression of cyclophilin.

34. A method of treating a cyclophilin mediated disease or injury in a mammal comprising administering a therapeutic effective amount of the compound of claim 4 to the mammal under conditions to treat the cyclophilin mediated disease or injury, wherein the disease or injury is one or both of mediated by the over expression of cyclophilin and a congenital over expression of cyclophilin.

35. A method of treating a cyclophilin mediated disease or injury in a mammal comprising administering a therapeutic effective amount of the compound of claim 1 to the mammal under conditions to treat the cyclophilin mediated disease or injury, wherein the disease or injury is selected from the group consisting of
a. a viral infection, wherein the viral infection is caused by a virus selected from the group consisting of Human Immunodeficiency virus, Hepatitis A, Hepatitis B, Hepatitis C, Hepatitis D, and Hepatitis E;
b. inflammatory disease, wherein the inflammatory disease is selected from the group consisting of asthma, autoimmune disease, chronic inflammation, chronic prostatitis, glomerulonephritis, hypersensitivity disease, inflammatory bowel disease, sepsis, vascular smooth muscle cell disease, aneurysms, pelvic inflammatory disease, reperfusion injury, rheumatoid arthritis, transplant rejection, and vasculitis;
c. cancer, wherein the cancer is selected from the group consisting of small and non-small cell lung, bladder, hepatocellular, pancreatic and breast cancer;
d. muscular degenerative disorder, wherein the muscular degenerative disorder is selected from the group consisting of myocardial reperfusion injury, muscular dystrophy, and collagen VI myopathies;
e. neurodegenerative disorder, wherein the neurodegenerative disorder is selected from the group consisting of Alzheimer's disease, Parkinson's disease, Huntington's disease, Multiple Systems Atrophy, Multiple Sclerosis, cerebral palsy, stroke, diabetic neuropathy, amyotrophic lateral sclerosis (Lou Gehrig's Disease), spinal cord injury, and cerebral injury; and
f. injury associated with loss of cellular calcium homeostasis, wherein the injury associated with loss of cellular calcium homeostasis is selected from the group consisting of myocardial infarct, stroke, acute hepatotoxicity, cholestasis, and storage/reperfusion injury of transplant organs.

36. A method of treating a cyclophilin mediated disease or injury in a mammal comprising administering a therapeutic effective amount of the compound of claim 2 to the mammal under conditions to treat the cyclophilin mediated disease or injury, wherein the disease or injury is selected from the group consisting of:
a. a viral infection, wherein the viral infection is caused by a virus selected from the group consisting of Human Immunodeficiency virus, Hepatitis A, Hepatitis B, Hepatitis C, Hepatitis D, and Hepatitis E;
b. inflammatory disease, wherein the inflammatory disease is selected from the group consisting of asthma, autoimmune disease, chronic inflammation, chronic prostatitis, glomerulonephritis, hypersensitivity disease, inflammatory bowel disease, sepsis, vascular smooth muscle cell disease, aneurysms, pelvic inflammatory disease, reperfusion injury, rheumatoid arthritis, transplant rejection, and vasculitis;
c. cancer, wherein the cancer is selected from the group consisting of small and non-small cell lung, bladder, hepatocellular, pancreatic and breast cancer;
d. muscular degenerative disorder, wherein the muscular degenerative disorder is selected from the group consisting of myocardial reperfusion injury, muscular dystrophy, and collagen VI myopathies;
e. neurodegenerative disorder, wherein the neurodegenerative disorder is selected from the group consisting of Alzheimer's disease, Parkinson's disease, Huntington's disease, Multiple Systems Atrophy, Multiple Sclerosis, cerebral palsy, stroke, diabetic neuropathy, amyotrophic lateral sclerosis (Lou Gehrig's Disease), spinal cord injury, and cerebral injury; and
f. injury associated with loss of cellular calcium homeostasis, wherein the injury associated with loss of cellular calcium homeostasis is selected from the group consisting of myocardial infarct, stroke, acute hepatotoxicity, cholestasis, and storage/reperfusion injury of transplant organs.

37. A method of treating a cyclophilin mediated disease or injury in a mammal comprising administering a therapeutic effective amount of the compound of claim 4 to the mammal under conditions to treat the cyclophilin mediated disease or injury, wherein the disease or injury is selected from the group consisting of
a. a viral infection, wherein the viral infection is caused by a virus selected from the group consisting of Human Immunodeficiency virus, Hepatitis A, Hepatitis B, Hepatitis C, Hepatitis D, and Hepatitis E;
b. inflammatory disease, wherein the inflammatory disease is selected from the group consisting of asthma, autoimmune disease, chronic inflammation, chronic prostatitis, glomerulonephritis, hypersensitivity disease, inflammatory bowel disease, sepsis, vascular smooth muscle cell disease, aneurysms, pelvic inflammatory disease, reperfusion injury, rheumatoid arthritis, transplant rejection, and vasculitis;
c. cancer, wherein the cancer is selected from the group consisting of small and non-small cell lung, bladder, hepatocellular, pancreatic and breast cancer;
d. muscular degenerative disorder, wherein the muscular degenerative disorder is selected from the group consisting of myocardial reperfusion injury, muscular dystrophy, and collagen VI myopathies;
e. neurodegenerative disorder, wherein the neurodegenerative disorder is selected from the group consisting of Alzheimer's disease, Parkinson's disease, Huntington's disease, Multiple Systems Atrophy, Multiple Sclerosis, cerebral palsy, stroke, diabetic neuropathy, amyotrophic lateral sclerosis (Lou Gehrig's Disease), spinal cord injury, and cerebral injury; and
f. injury associated with loss of cellular calcium homeostasis, wherein the injury associated with loss of cellular calcium homeostasis is selected from the group consisting of myocardial infarct, stroke, acute hepatotoxicity, cholestasis, and storage/reperfusion injury of transplant organs.
